SEQUENCE LISTING (17 sequences)

SEQ ID NO: 1 - DNA, 19 bp, Artificial Sequence
  primer 1
ggttcctattgggcttgct

SEQ ID NO: 2 - DNA, 20 bp, Artificial Sequence
  primer 2
ccaccctcattttcagggat

SEQ ID NO: 3 - DNA, 20 bp, Artificial Sequence
  FRET labeled primer
  misc_feature (1)..(1): 5′-(BHQ1) label
  misc_feature (13)..(13): FAM-U
ccaccctcatttncagggat

SEQ ID NO: 4 - DNA, 39 bp, Artificial Sequence
  fragment of target sequence M13mp18
ggttcctattgggcttgctatccctgaaaatgagggtgg

SEQ ID NO: 5 - DNA, 40 bp, Artificial Sequence
  first modified fragment of target sequence M13mp18
ggttcctattgggcttgctaatccctgaaaatgagggtgg

SEQ ID NO: 6 - DNA, 42 bp, Artificial Sequence
  second modified fragment of target sequence M13mp18
ggttcctattgggcttgctaaaatccctgaaaatgagggtgg

SEQ ID NO: 7 - DNA, 44 bp, Artificial Sequence
  third modified fragment of target sequence M13mp18
ggttcctattgggcttgctaaaaaatccctgaaaatgagggtgg

SEQ ID NO: 8 - DNA, 47 bp, Artificial Sequence
  fourth modified fragment of target sequence M13mp18
ggttcctattgggcttgctaaaaaaaaatccctgaaaatgagggtgg

SEQ ID NO: 9 - DNA, 47 bp, Artificial Sequence
  fifth modified fragment of target sequence M13mp18
ggttcctattgggcttgctatatatatatccctgaaaatgagggtgg

SEQ ID NO: 10 - DNA, 51 bp, Artificial Sequence
  sixth modified fragment of target sequence M13mp18
ggttcctattgggcttgctaaaaaaaaaaaaatccctgaaaatgagggtgg

SEQ ID NO: 11 - DNA, 51 bp, Artificial Sequence
  seventh modified fragment of target sequence M13mp18
ggttcctattgggcttgctatatatatatatatccctgaaaatgagggtgg

SEQ ID NO: 12 - DNA, 40 bp, Artificial Sequence
  eigth modified fragment of target sequence M13mp18
ggttcctattgggcttgctgatccctgaaaatgagggtgg

SEQ ID NO: 13 - DNA, 41 bp, Artificial Sequence
  ninth modified fragment of target sequence M13mp18
ggttcctattgggcttgctggatccctgaaaatgagggtgg

SEQ ID NO: 14 - DNA, 34 bp, Artificial Sequence
  nucleotide-modified primer
  modified_base (1)..(34): A at positions 1, 5, 11, 16 and 18 are 2,6- diaminopurine; T at positions 3, 7, 9, 10, 15, 19, 23, 24, 27, 29, 30 are 5-propynyl 2′-deoxyuridine; and C at positions 2, 4, 12, 17, 25, 26, 34 are 5-propynyl 2′-deoxycytidine
actcagtgttacggtacatgggttcctattgggc

SEQ ID NO: 15 - DNA, 15 bp, Artificial Sequence
  FRET probe
  misc_feature (1)..(1): 5′-FAM label
  misc_feature (15)..(15): (BHQ1)-3′ label
ttcagggatagcaag

SEQ ID NO: 16 - DNA, 20 bp, Artificial Sequence
  primer 16
tcagagccaccaccctcatt

SEQ ID NO: 17 - DNA, 86 bp, Artificial Sequence
  second fragment of target sequence M13mp18
ggtgacgaaactcagtgttacggtacatgggttcctattgggcttgctatccctgaaaatgagggtggtggctctgagggtggcgg

(19) United States
(12) Patent Application Publication
(10) Pub. No.: US 2021/0388431 A1
(43) Pub. Date: Dec. 16, 2021

(54) THERMOKINETICALLY BALANCED ISOTHERMAL AMPLIFICATION OF NUCLEIC ACID SEQUENCES

(71) Applicant: Igor V. Kutyavin, Woodinville, WA (US)

(72) Inventor: Igor V. Kutyavin, Woodinville, WA (US)

(21) Appl. No.: 17/290,186

(22) PCT Filed: Oct. 28, 2019
(86) PCT No.: PCT/US2019/058399

§ 371 Date: Apr. 29, 2021

Related U.S. Application Data
(60) Provisional application No. 62/752,283, filed on Oct. 29, 2018.
(60) Provisional application No. 62/843,193, filed on May 3, 2019.

(51) Int. Cl. C12Q 1/6853; C12Q 1/686; C12P 19/34; C12Q 1/6876

(52) U.S. Cl. CPC C12Q 1/6853; C12Q 1/6876; C12P 19/34; C12Q 1/686

(57) ABSTRACT

Provided are methods for isothermal amplification of nucleic acids wherein hybridization of one or both target-amplifying primers to corresponding target sequence strands, primer extension by a DNA polymerase and denaturation of the resulting target sequence amplicons takes place at the same temperature in an isothermal cycling mode, thus amplifying the target nucleic acid sequence. The methods were shown to substantially accelerate conventional PCR. Also provided are kits comprising at least one, preferably at least two target-specific oligonucleotides configured to provide for accelerated isothermal amplification.

### INCORPORATION OF SEQUENCE LISTING

The content of the text file named “0067898_014WO0_ST25.txt,” which was created on Oct. 28, 2019, and is 4.33 KB in size, is hereby incorporated by reference in its entirety.

### FIELD OF THE INVENTION

Particular aspects of the invention relate to methods for isothermal amplification of nucleic acids, and more particularly to thermokinetically balanced isothermal amplification methods wherein hybridization of oligonucleotide primer(s) to target nucleic acid template strand(s), extension of the primer(s) by a DNA polymerase and denaturation of the primer-extension products all occur isothermally at the same reaction temperature. Additional aspects relate to PCR methods comprising the thermokinetically balanced isothermal amplification methods, and kits for carrying out the thermokinetically balanced isothermal amplification methods, etc.

### BACKGROUND

Polymerase chain reaction (PCR) (see, e.g., U.S. Pat. Nos. 4,683,195 and 4,683,202 to Mullis K. B., 1987) continues to be the most commonly used technology for amplification of nucleic acids in research laboratories as well as in commercial applications. PCR is based on repetitive temperature changes during the amplification process. Several isothermal amplification methods have been developed which, unlike PCR, does not require temperature changes during the amplification (temperature cycling), and which may rather be conducted at a relatively constant temperature. Examples of these isothermal amplification technologies include NASBA (e.g., U.S. Pat. No. 6,063,603), HDA (e.g., Vincent M. et al, 2004), Rolling Circle Amplification (e.g., U.S. Pat. Nos. 5,854,033 and 6,210,884 to Lizardi P., 1998 and 2001), Loop-mediated isothermal amplification (e.g., U.S. Pat. No. 6,410,278 to Notomi T. and Hase T., 2002), amplification methods based on the use of RNA or composite RNA/DNA primers (e.g., U.S. Pat. No. 5,824,517 to Cleuziat P. and Mandrand B., 1998; U.S. Pat. No. 6,251,639 to Kurn N., 2001), Strand Displacement Amplification (e.g., U.S. Pat. No. 5,270,184 to Walker G. T. et al, 1993; U.S. Pat. No. 5,648,211 to Fraiser M. S. et al, 1997; U.S. Pat. No. 5,712,124 to Walker G. T., 1998), Nick Displacement Amplification (PCT Patent Application WO 2006/125267 to Millar D. S. et al, 2006; U.S. Patent Application Publication 2003/0138800 to Van Ness J. et al, 2003), Accelerated Cascade Amplification (PCT Patent Application WO/2008/086381 to Nelson J. R. et al, 2008; U.S. Pat. No. 8,143,006 to Kutyavin I. V., 2012) and other techniques.

Although a variety of the amplification protocols has been discovered and developed to date (see examples above), there is still a pronounced need in the art for more efficient, sequence specific and sensitive, fast and versatile methods of nucleic acids amplification and detection.

### BRIEF DESCRIPTION OF THE DRAWINGS

FIG. 1 schematically shows, by way of non-limiting examples of the present invention, a first embodiments of an isothermal amplification method for generation of multiple copies of a second strand of a target nucleic acid sequence (e.g., DNA), wherein a first strand of the target sequence includes a 5′-end of a template polynucleotide, wherein the template polynucleotide may consist of, or be longer than the target sequence (i.e., the template polynucleotide may extend a known distance beyond the target sequence in the 3′-direction, or have an indefinite 3′-end). At a P1 primer cycling temperature, complementary oligonucleotide primer P1 hybridizes to the first strand of the target sequence (step A), and is extended to the 5′-end of the target sequence by a DNA polymerase to provide a hybridized P1 primer extension product (step B), which denatures from the first strand of the target sequence to provide a non-hybridized copy of the second strand of the target sequence and the template polynucleotide in P1-primable form. The reaction mixture contains all reagents necessary for DNA synthesis including DNA polymerase and 2′-deoxynucleoside 5′-triphosphates (dNTPs) and is incubated at the P1 primer cycling temperature that is sufficient to support multiple isothermal cycles of all three reaction steps. This enables cycling of the amplification reaction, and production of multiple copies of the second strand of the target sequence primer extension products (second strand of the target nucleic acid) from the same first strand target sequence molecule (or template oligonucleotide) as illustrated in FIG. 1.

FIG. 2 schematically shows, by way of non-limiting examples of the present invention, a second isothermal amplification method embodiment for generation of multiple copies of a target nucleic acid sequence (e.g., DNA). The method is an extension of the method of FIG. 1, involving use of two primers, P1 and P2 each having complementary binding sites on different strands of the target sequence, wherein the extension product of each primer serves as a template for the other primer, and wherein the reaction is incubated at a primer cycling temperature that supports isothermal cycling through steps A-C for primer P1, and that also supports isothermal cycling through steps A-C for primer P2. The isothermal amplification(s) can be triggered by addition to the reaction mixture of either strand, or both strands of the target sequence that includes the 5′-end of a template polynucleotide, which may consist of, or be longer than the target sequence (i.e., the template polynucleotide may extend a known distance beyond the target sequence in the 3′-direction, or have an indefinite 3′-end). Preferably, the template polynucleotide for P1 isothermal cycling consists of a complementary first target sequence strand (e.g., consists of a P2 primer extension product), and the template polynucleotide for P2 isothermal cycling consists of a complementary second target sequence strand (e.g., consists of a P1 primer extension product).

FIG. 3 schematically shows, by way of non-limiting examples of the present invention, a third thermal cycling amplification embodiment for generation of multiple copies of a target nucleic acid sequence (e.g., DNA). Like the method of FIG. 2, the method of FIG. 3 is an extension of the method of FIG. 1, involving use of two primers, P1 and P2 each having complementary binding sites on different strands of the target sequence, wherein the extension product of each primer may serve as a template for the other primer, but unlike in the method of FIG. 2, the P2 primer has weaker hybridization properties with its binding site than does the P1 primer with its binding site, and thus when the reaction temperature is held at the P1 primer cycling temperature (“Temperature 1”), the P2 primer is incapable of (or inefficient at) hybridizing to the P1 primer extension product to produce a P2 primer extension product. However, as shown in the lower half of FIG. 3, the temperature of the reaction can be reduced to a lower temperature (“Temperature 2”) that supports P2 primer hybridization to the P1 primer extension products generated and accumulated at Temperature 1, and extension to produce stable double-stranded target sequences (i.e., P2 primer extension products that remain hybridized to the accumulated P1 primer extension products at Temperature 2). Returning the reaction temperature to Temperature 1 initiates denaturation of the double-stranded target sequences produced and accumulated at Temperature 2, thereby introducing many copies of single-stranded template for cycling with P1 primers at the P1 primer cycling temperature (Temperature 1). Somewhat similar to a traditional PCR reaction, the method of FIG. 3 involves cycling between two temperatures (Temperature 1 and Temperature 2). Unlike classical PCR, however, the incubation at Temperature 1 is not merely to initiate melting of a target amplicon to provide a single-stranded template for primer P1, but also supports isothermal cycling through steps A (P1 primer hybridization), B (P1 primer extension), and C (denaturation) to produce and accumulate multiple P1 primer extension products from each single-stranded P1 template, including those single-stranded P1 templates that are P2 primer extension products produced by denaturing the stable target sequence duplexes accumulated at Temperature 2. The thermal cycling amplification embodiment, comprising an isothermal amplification stage (Temperature 1), can be triggered by addition to the reaction mixture of the same target sequences as described in FIG. 2.

FIG. 4 shows, by way of non-limiting examples of the present invention, a fragment of a M13mp18 vector sequence (target DNA, SEQ ID NO:4) and two, 2′-deoxyribo oligonucleotide primers (SEQ ID NOS:1 and 2) used in the working Examples provided herein. Oligonucleotide SEQ ID NO:3 is a FRET-modified primer that is an analog of primer SEQ ID NO:2 incorporating a 5′-conjugated Black Hole Quencher™ dye (BHQ1) and 6-fluorescein dye (FAM) that was internally linked to the 5 base position of deoxyribo uridine nucleoside (shown as boldface “U”). The oligonucleotide sequences are aligned with the target DNA in the 5′-to-3′ orientation as indicated. 2′-Deoxyribo oligonucleotides SEQ ID NOS:5-13 are variants of SEQ ID NO:4 incorporating artificial nucleotide insertions of different length and composition (boldface type and underlined) between the forward primer sequence (SEQ ID NO:1) and the binding site of the reverse primer (SEQ ID NO:2), in each case within the shown SEQ ID NO:4 strand. These oligonucleotides were used as target sequences in the experiments giving rise to the data shown in FIG. 6.

FIGS. 5A and 5B show, by way of non-limiting examples of the present invention, results of variable target loads of template oligonucleotide SEQ ID NO:4 using primers SEQ ID NOS:1 and 2 (see structures in FIG. 4) obtained by EvaGreen™ fluorescence monitoring during isothermal amplification at a P1 and P2 primer cycling temperature of 74° C., using the thermokinetically-balanced isothermal amplification reaction scheme shown in FIG. 2. The fluorescence threshold for each curve of FIG. 5A was determined (at dashed line) and plotted versus the logarithm of the target loads in FIG. 5B. The slope coefficient of the linear equations indicates that the amount of the amplified target material in the reaction mixture is increased by an order of magnitude every ˜27 seconds.

FIG. 6 shows, by way of non-limiting examples of the present invention, results of EvaGreen™ fluorescence monitoring during isothermal amplification (74° C.), using the reaction scheme shown in FIG. 2, of target sequences incorporating nucleotide insertions of variable length and composition between the forward primer sequence (SEQ ID NO:1) and the reverse primer (SEQ ID NO:2) binding site (in each case within the SEQ ID NO:4 strand shown in FIG. 4). The target sequences and primers are shown in FIG. 4. For convenience, sequences of the insertions are shown in brackets next to the oligonucleotide number in FIG. 6.

FIGS. 7A and 7B show, by way of non-limiting examples of the present invention, results of fluorescence monitoring during isothermal amplification (73° C.), using the reaction scheme shown in FIG. 2, of variable target loads of oligonucleotide SEQ ID NO:4 using primers SEQ ID NOS:1 and 3 (see structures in FIG. 4). This experiment is similar to that shown in FIG. 5, but EvaGreen™ dye was omitted and the fluorescence signal was provided by a FRET-effect of the dyes-labeled reverse primer SEQ ID NO:3. The fluorescence threshold for each curve of FIG. 7A was determined (at dashed line) and plotted versus the logarithm of the target loads in FIG. 7B. The slope coefficient of the linear equations indicates that the amount of the amplified target material in the reaction mixture is increased by an order of magnitude every ˜62 seconds.

FIG. 8 shows, by way of non-limiting examples of the present invention, results of EvaGreen™ fluorescence monitoring during isothermal amplification (74° C.), using the reaction scheme shown in FIG. 2. The experiment of FIG. 8 is similar to that shown in FIG. 5A in many aspects including oligonucleotide primers used (SEQ ID NOS:1 and 2) and concentration of the reaction components, but with one key difference. A double-stranded circular cloning vector M13mp18 (7249-base pair long polymer) was linearized by restriction enzyme cleavage as described herein under working Example 2. The linearized M13mp18 vector was then used, as shown in FIG. 8, as an amplification-triggering DNA polynucleotide (10,000 molecules per a reaction) incorporating the target sequence, i.e., the sequence of oligonucleotide SEQ ID NO:4 used in the experiments of FIG. 5A for the same purpose. In one of the time/temperature profiles of FIG. 8 (see dashed line profile), after mixing all components, the reaction was kept isothermal at 74° C. with florescence monitored every 10 seconds. The other two profiles followed one cycle (middle profile) or two cycles (left-most profile) of a two-step incubation comprising an initial 15 second incubation at 95° C., followed by an isothermal amplification step at 74° C.

FIGS. 9A and 9B show, by way of non-limiting examples of the present invention, results of fluorescence monitoring during PCR using a forward primer (SEQ ID NO:1), and a FRET-labeled reverse primer (SEQ ID NO:3, see structures in FIG. 4). Shown in FIGS. 9A and 9B are two groups of curves, each group labeled by a different marker. The fluorescence curves labeled by empty circles (o) correspond to a conventional PCR format using cycles having only two steps (primer-annealing and amplicon-denaturing), whereas the curves labeled by filled circles/dots (•) relate to the thermokinetically-balanced isothermal amplification reaction scheme shown in FIG. 2, having cycles further comprising an isothermal primer cycling step (i.e. incubation of the reaction at a primers-cycling temperature of 73° C. for 20 seconds). The four curves within each group differ only by the respective target reaction load (linearized, double-stranded cloning vector M13mp18 as described in Example 2) that changes by four orders of magnitude from left to right as indicated. Detailed description of the experiments is provided herein in working Example 3.5. The fluorescence threshold for the two curve groups of FIG. 9A was determined (dashed line) and plotted versus the logarithm of the target loads in FIG. 9B. The data points of FIG. 9B display good linear trends with R² value >0.99. The slopes of the linear equations were used to calculate a PCR amplification power coefficient for each case. These coefficients, rounded to tenths, as well as the linear equations used for calculation (FIG. 9B), primers and abbreviation of PCR time/temperature profiles used in the reactions are listed at the top of FIGS. 9A and 9B identified by corresponding symbol. For example, time/temperature profile abbreviation 95° 15″→(95°5″→62°10″→73°20″)₄₀ means that the reaction mixture was initially incubated at 95° C. for 15 second, followed by 40 cycles each comprising incubation at 95° C. for 5 seconds, incubation at 62° C. for 10 seconds, followed by incubation for 20 seconds at 73° C. (the “Primers-cycling temperature”). The fluorescence detection stage is underlined.

FIG. 10 shows, by way of non-limiting examples of the present invention, a fragment of a M13mp18 vector sequence (target DNA SEQ ID NO:17), two 2′-deoxyribo oligonucleotide primers (SEQ ID NOS:14 and 16) and a 5′-nuclease-clevable FRET probe (SEQ ID NO:15) that were used in the experiments of FIG. 11. Forward primer SEQ ID NO:14 contains base-modified duplex-stabilizing nucleotide analogs 5-propynyl 2′-deoxyuridine, 5-propynyl 2′-deoxycytidine and 2,6-diaminopurine nucleotide shown respectively as boldface T, C and A. Oligonucleotide SEQ ID NO:15 is a FRET-labeled probe incorporating a 3′-conjugated Black Hole Quencher™ dye (BHQ1) and 5′-conjugated 6-fluorescein dye (FAM). The oligonucleotide sequences are aligned with the target DNA in the 5′-to-3′ orientation as indicated.

FIGS. 11A and 11B show, by way of non-limiting examples of the present invention, results of fluorescence monitoring during PCR using forward (SEQ ID NO:14) and reverse primer (SEQ ID NO:16) and a 5′-nuclease cleavable FRET-probe (SEQ ID NO:15, see structures in FIG. 10). Shown in FIGS. 11A and 11B are two groups of curves, each group labeled by respective symbols. The fluorescence curves labeled by empty circles (o) correspond to a conventional PCR format using cycles having only two steps (primer-annealing and amplicon-denaturing steps), whereas the curves labeled by filled circles/dots (•) are derived using a PCR format incorporating the method embodiment of FIG. 3, with each cycle further comprising an isothermal forward-primer cycling step, (i.e., incubation of the reaction at the forward “Primer-cycling temperature” of 85° C. for 40 seconds). The five curves within each group differ only by the target reaction load (linearized double-stranded cloning vector M13mp18, as described in Example 2) that differs by five orders of magnitude from left to right as indicated. A detailed description of the experiments is provided herein in working Example 3.6. The fluorescence threshold for the two curve groups of FIG. 11A was determined (dashed line) and plotted versus the logarithm of the target loads in FIG. 11B. The slopes of the linear equations were used to calculate a PCR amplification power coefficient for each group. The coefficients, rounded to tenths, as well as the linear equations used for calculation (FIG. 11B), primers and abbreviation of PCR time/temperature profiles used in the reactions are listed at the top of FIGS. 11A and 11B identified by corresponding symbol.

### SUMMARY OF EXEMPLARY EMBODIMENTS OF THE INVENTION

Embodiments of the disclosure can be described in view of the following clauses:

1. A method for isothermally-accelerated amplification of a target nucleic acid sequence, comprising:
incubating a reaction mixture at a primer-cycling temperature, the reaction mixture sufficient to support DNA synthesis and containing DNA polymerase activity, complementary first and second target sequence template strands, a first primer P1 complementary to a 3′-terminal portion of the first target sequence template strand, a second oligonucleotide primer P2 complementary to a 3′-terminal portion of the second target sequence template strand, the P1 and P2 primers each present in excess molar concentration relative to the first and second target sequence template strands, respectively;
hybridizing, during the incubating, P1 and P2 primers to the first and the second target sequence template strands, respectively;
extending, during the incubating, the hybridized P1 and P2 primers to produce second and first target sequence template strands, respectively;
denaturing, during the incubating, the first and the second target sequence template strands to provide the first and the second target sequence template strands in P1- and P2-primable form, respectively; and
cyclically repeating, during the incubating, the hybridizing, extending and denaturing steps for the P1 and P2 primers isothermally at the primer-cycling temperature to provide isothermal P1 and P2 primer-driven cycling, wherein in each consecutive P1 and P2 isothermal cycle, at least some of the respective second and first target sequence template strands produced in and accumulated over all prior isothermal cycles serve as additional second and first target sequence template strands, to provide for isothermally-accelerated amplification of the target nucleic acid sequence.
2. The method of clause 1, wherein the isothermal cycles for the P1 and P2 primers are symmetric, or substantially symmetric, such that the number of first and second target sequence template strands produced and accumulated is equal or substantially equal.
3. The method of clause 1, wherein isothermal cycles for the P1 and P2 primers are, at least to some extent asymmetric, such that the number of first and second target sequence template strands produced and accumulated at one or more incubation times during the reaction is not equal.
4. The method of clause 3, comprising increasing or decreasing the asymmetry by varying the relative concentrations of the P1 and the P2 primers.
5. A method for producing multiple copies of a target nucleic acid sequence, comprising:
incubating a reaction mixture at a P1 primer-cycling temperature (P1-PCT), the reaction mixture sufficient to support DNA synthesis and containing DNA polymerase activity, a first target sequence template strand, a first primer P1 complementary to a 3′-terminal portion of the first target sequence template strand and present in excess molar concentration relative to the first target sequence template strand;
hybridizing, during the incubating at the P1-PCT, a P1 primer to the first target sequence template strand;
extending, during the incubating at the P1-PCT, the hybridized P1 primer to produce a complementary second target sequence template strand having a P2 primer-binding site at a 3′-terminal portion thereof;
denaturing, during the incubating at the P1-PCT, the first and the second target sequence template strands to provide the first and the second target sequence template strands in P1- and P2-primable form, respectively; and cyclically repeating, during the incubating, the hybridizing, extending and denaturing steps isothermally at the P1-PCT, to provide isothermal P1 primer-driven cycling to isothermally produce multiple copies of the second target sequence template strand in P2-primable form.
6. The method of clause 5, wherein the reaction mixture contains a second primer P2 complementary to the 3′-terminal portion of the second target sequence template strand and present in excess molar concentration relative to the second target sequence template strand, and wherein the method comprises:
incubating the reaction mixture at the P1-PCT;
hybridizing, during the incubating at the P1-PCT, P1 and P2 primers to the first and the second target sequence template strands, respectively;
extending, during the incubating at the P1-PCT, the hybridized P1 and P2 primers to produce second and first target sequence template strands, respectively;
denaturing, during the incubating at the P1-PCT, the first and the second target sequence template strands to provide the first and the second target sequence template strands in P1- and P2-primable form, respectively; and cyclically repeating, during the incubating, the hybridizing, extending and denaturing steps isothermally at the P1-PCT to provide isothermal P1 and P2 primer-driven cycling, wherein in each consecutive P1 and P2 isothermal cycle, at least some of the respective second and first target sequence template strands produced in and accumulated over all prior isothermal cycles serve as additional second and first target sequence template strands, to provide for isothermally-accelerated amplification of the target nucleic acid sequence.
7. The method of clause 6, wherein the P1 and P2 isothermal cycles are symmetric, or substantially symmetric, such that the number of first and second target sequence template strands produced and accumulated is equal or substantially equal.
8. The method of clause 6, wherein the P1 and P2 isothermal cycles are, at least to some extent, asymmetric, such that the number of first and second target sequence template strands produced and accumulated at one or more incubation times during the reaction is not equal.
9. The method of clause 8, comprising increasing or decreasing the asymmetry by varying the relative concentrations of the P1 and the P2 primers.
10. The method of clause 5-9, wherein the reaction mixture contains a second primer P2 complementary to the 3′-terminal portion of the second target sequence template strand and present in excess molar concentration relative to the second target sequence template strand, and wherein the reaction further comprises, after the repeating to provide the isothermal P1 primer-driven cycling,
incubating the reaction mixture at a P2 primer hybridization and extension temperature (P2-PHET) lower than the P1-PCT;
hybridizing, during the incubating at the P2-PHET, P2 primers to the second target sequence template strands produced at the P1-PCT; and
extending, during the incubating at the P2-PHET, the hybridized P2 primers to produce complementary first target sequence template strands hybridized to the second target sequence template strands produced at the P1-PCT.
11. The method of clause 10, comprising, after extending at the P2-PHET, incubating the reaction mixture at the P1-PCT.
12. The method of clause 11, comprising alternating the incubation temperature between the P1-PCT and the P2-PHET to provide alternating P1-PCT and P2-PHET stages, and wherein in each consecutive stage at least some of the respective second and first target sequence template strands produced in and accumulated over all prior stages serve as additional second and first target sequence template strands, to provide for isothermally-accelerated amplification of the target nucleic acid sequence.
13. The method of clause 12, wherein after extending at the P2-PHET, incubating the reaction mixture at the P1-PCT denatures the hybridized template strands produced at the P2-PHET to provide the first and the second target sequence template strands in P1- and P2-primable form, respectively.
14. The method of clause 12 or 13, wherein alternating the reaction temperature between the P1-PCT and the P2-PHET to provide alternating P1-PCT and P2-PHET stages comprises, before or after incubating at the P1-PCT, incubating at a denaturation acceleration temperature greater than the P1-PCT to facilitate denaturation of the hybridized template strands produced at the P2-PHET.
15. A method for isothermally-accelerated amplification of a target nucleic acid sequence, comprising:
incubating a reaction mixture at a P1-primer-cycling temperature (P1-PCT), the reaction mixture sufficient to support DNA synthesis and containing DNA polymerase activity, complementary first and second target sequence template strands, a first primer P1 complementary to a 3′-terminal portion of the first target sequence template strand, a second oligonucleotide primer P2 complementary to a 3′-terminal portion of the second target sequence template strand, the P1 and P2 primers each present in excess molar concentration relative to the first and second target sequence template strands, respectively;
hybridizing, during the incubating at the P1-PCT, a P1 primer to the first target sequence template strand;
extending, during the incubating at the P1-PCT, the hybridized P1 primer to produce a complementary second target sequence template strand having a P2 primer-binding site at a 3′-terminal portion thereof;
denaturing, during the incubating at the P1-PCT, the first and the second target sequence template strands to provide the first and the second target sequence template strands in P1- and P2-primable form, respectively; and
repeating, during the incubating, the hybridizing, extending and denaturing steps isothermally at the P1-PCT, to provide isothermal P1 primer-driven cycling to isothermally produce multiple copies of the second target sequence template strand in P2-primable form;
incubating, after the P1 primer-driven cycling, the reaction mixture at a P2 primer hybridization and extension temperature (P2-PHET) lower than the P1-PCT; hybridizing, during the incubating at the P2-PHET, P2 primers to the second target sequence template strands produced at the P1-PCT;
extending, during the incubating at the P2-PHET, the hybridized P2 primers to produce complementary first target sequence template strands hybridized to the second target sequence template strands produced at the P1-PCT;
incubating, after extending at the P2-PHET, the reaction mixture at the P1-PCT; and
alternating the reaction temperature between the P1-PCT and the P2-PHET to provide alternating P1-PCT and P2-PHET stages, and wherein in each consecutive stage at least some of the respective second and first target sequence template strands produced in and accumulated over all prior stages serve as additional second and first target sequence template strands, to provide for isothermally-accelerated amplification of the target nucleic acid sequence.
16. The method of clause 15, wherein after extending at the P2-PHET, incubating the reaction mixture at the P1-PCT denatures the hybridized template strands produced at the P2-PHET to provide the first and the second target sequence template strands in P1- and P2-primable form, respectively.
17. The method of clause 15 or 16, wherein alternating the reaction temperature between the P1-PCT and the P2-PHET to provide alternating P1-PCT and P2-PHET stages comprises, before or after incubating at the P1-PCT, incubating at a denaturation acceleration temperature greater than the P1-PCT to facilitate denaturation of the hybridized template strands produced at the P2-PHET.
18. The method of any one of clauses 1-17, present as an isothermal acceleration step of a PCR reaction.
19. A PCR reaction comprising at least one cycle having an isothermal amplification step according to claims 1-17.
20. The method of any one of clauses 1-19, wherein the primer(s) that provide isothermal primer-driven cycling are used at a reaction concentration greater than 200 nanomolar.
21. The method of any one of clauses 1-20, wherein the P1 or the P2 primer or both primer sequences incorporate at least one DNA polymerase-compatible structural modification.
22. The method of any one of clauses 5, 10-17, wherein the P1 primer incorporates at least one polymerase-compatible duplex-stabilizing structural modification.
23. The method of any one of clauses 1-22, wherein the amplification products are detected.
24. The method of clause 23, wherein the amplification and detection reactions are performed simultaneously, in real time.
25. The method of clause 1-24, further comprising determining the amount of the target nucleic acid in or from a sample. 26. The method of clause 25, wherein the reaction mixture further comprises a detectable label.
27. The method of clause 26, wherein the detectable label comprises a fluorescent label.
28. The method of clause 27, wherein the reaction mixture comprises an oligonucleotide probe labeled with two dyes that are in FRET interaction, and wherein duplex formation of the probe with products of extension of first or second primers disrupts FRET resulting in a detectable signal.
29. The method of clause 27, wherein at least one of the P1 and P2 primers is labeled with two dyes that are in FRET interaction, and wherein hybridization and extension of the primer during the amplification disrupts FRET resulting in a detectable signal.
30. The methods of clauses 1-4, 6-9 and 18-29, wherein the distance, in nucleotides, between the 5′ end of first primer binding site on the first strand and the 5′ end of the second primer binding site on the second strand within the target sequence template strands is less than 20, less than 15, less than 10, less than 5, less than 4, less than 3, less than 2, 1, or 0, or is a value in the range of 0-20, or in any subrange thereof.
31. The method of any one of clauses 1-30, wherein the DNA polymerase activity is provided by one of Vent(exo-) and Deep Vent(exo-) DNA polymerases or a combination thereof.
32. An isothermally-accelerated amplification kit, comprising at least two oligonucleotide primers each complementary to a respective different primer binding site of a target sequence, wherein a first oligonucleotide primer is complementary to a first primer binding site on a first strand of the target sequence, wherein the second oligonucleotide primer is complementary to a second primer binding site on a second, complementary strand of the target sequence to define an amplicon bracketed by the first and second primers, and wherein, relative to the target sequence, the sequences and relative positions of the first and second primer binding sites on the target sequence are such that thermal stability of the primers and their extension products, when hybridized to the target sequence, provides for isothermal cycles of primer binding, primer extension, and primer extension product denaturation.
33. The kit of clause 32, wherein the distance, in nucleotides, between the 5′ end of first primer binding site on the first strand and the 5′ end of the second primer binding site on the second strand is less than 20, less than 15, less than 10, less than 5, less than 4, less than 3, less than 2, 1, or 0, or is a value in the range of 0-20, or in any subrange thereof.
34. The kit of clause 33, wherein the distance is 0 to 3 nucleotides.
35. An isothermally-accelerated amplification kit, comprising at least two oligonucleotide primers each complementary to a respective different primer binding site of a target sequence, wherein a first oligonucleotide primer is complementary to a first primer binding site on a first strand of the target sequence, wherein the second oligonucleotide primer is complementary to a second primer binding site on a second, complementary strand of the target sequence to define an amplicon bracketed by the first and second primers, and wherein, relative to the target sequence, the distance, in nucleotides, between the 5′ end of first primer binding site on the first strand and the 5′ end of the second primer binding site on the second strand is less than 20, less than 15, less than 10, less than 5, less than 4, less than 3, less than 2, 1, or 0, or is a value in the range of 0-20, or in any subrange thereof.
36. The kit of clause 35, wherein the distance is 0 to 3 nucleotides.
37. The kit of clause 35 or 36, wherein, relative to the target sequence, the sequences and relative positions of the first and second primer binding sites on the target sequence are such that thermal stability of the primers and their primer extension products, when hybridized to the target sequence, provides for isothermal cycles of primer binding, primer extension, and primer extension product denaturation.

### DETAILED DESCRIPTION OF EXEMPLARY EMBODIMENTS

Terms and symbols of biochemistry, nucleic acid chemistry, molecular biology and molecular genetics used herein follow those of standard treaties and texts in the field (e.g., Sambrook J. et al, 1989; Kornberg A. and Baker T., 1992; Gait M. J., ed., 1984; Lehninger A. L., 1975; Eckstein F., ed., 1991, and the like). To facilitate understanding of particular exemplary aspects of the invention, a number of terms are discussed below.

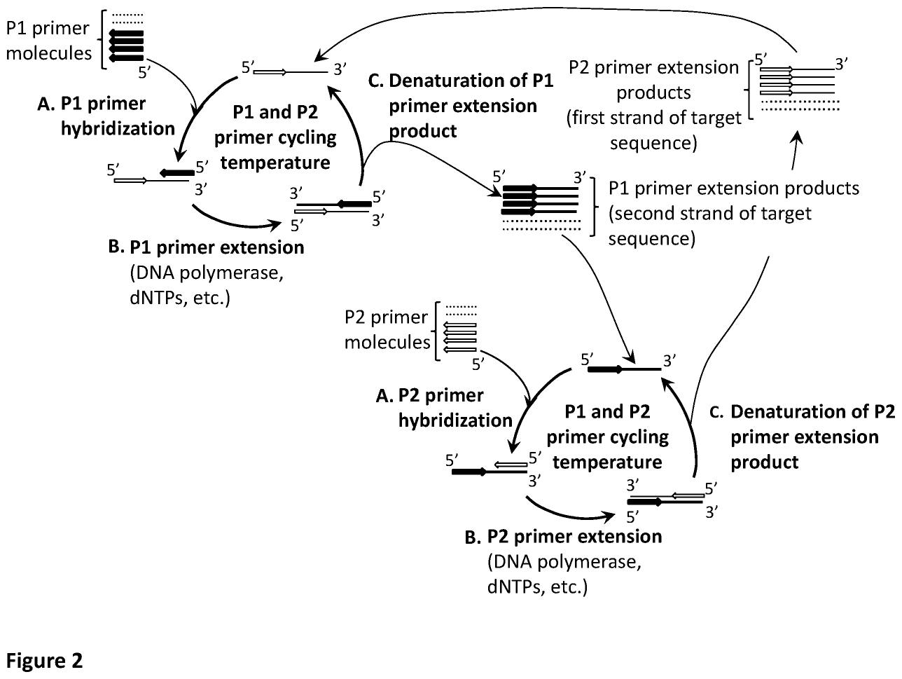

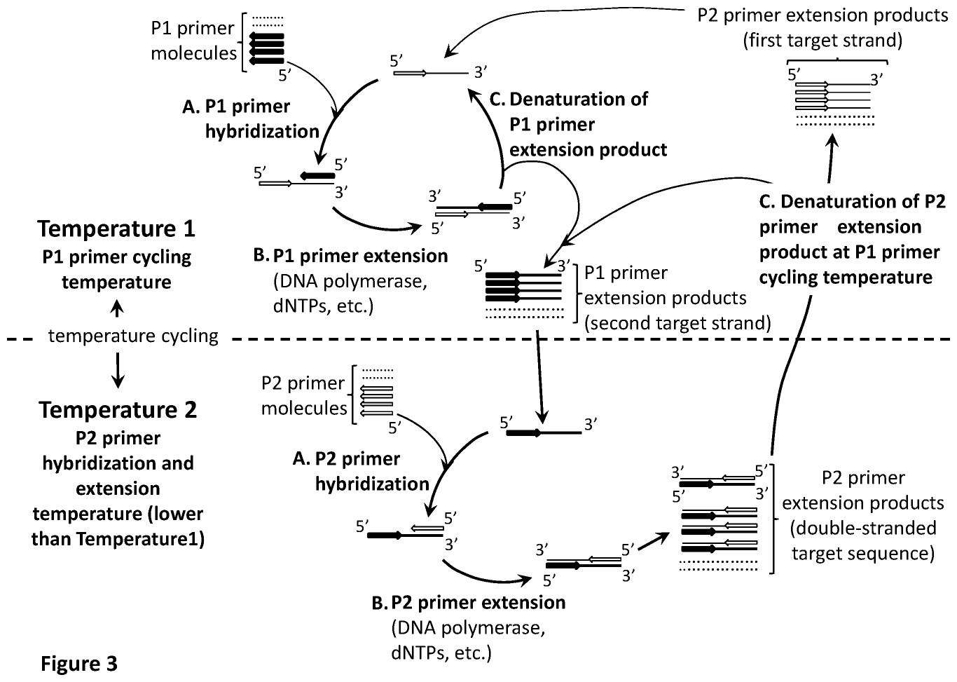

In the methods, “target nucleic acid” or “nucleic acid of interest” refers to a nucleic acid or a fragment or contiguous portion of nucleic that is to be amplified and/or detected using methods of the present invention. For example, the target nucleic acid sequence is framed by sequences and/or binding sites of P1 and P2 primers in methods of FIGS. 2 and 3. Nucleic acids of interest (e.g., target sequences) can be of any size and sequence. Preferably, the nucleic acid is of a size that provides for amplification and/or detection thereof. Two or more target nucleic acids can be fragments or portions (e.g., separated or contiguous portions) of the same nucleic acid molecule. As used herein, target nucleic acids are different if they differ in nucleotide sequence by at least one nucleotide. Target nucleic acids can be single-stranded or double-stranded. When a nucleic acid of interest is double-stranded or presumed to be double-stranded, the term “target nucleic acid” refers to a specific sequence in either strand of double-stranded nucleic acid. Therefore, the full complement to any single stranded nucleic acid of interest is treated herein as the same (or complementary) target nucleic acid. When a target nucleic acid comprises only one strand, the primers are preferably selected such as P1 primer hybridized to that single-stranded target sequence. When target nucleic acids are double-stranded, they are rendered single stranded by any physical, chemical or biological approach before applying the methods of the invention. For example, double-stranded nucleic acid can be denatured at elevated temperature, e.g. 90-95° C. as was used in the examples provided herein. Nucleic acids incorporating the target nucleic acids' sequences may be derived from any organism or other source, including but not limited to prokaryotes, eukaryotes, plants, animals, and viruses, as well as synthetic nucleic acids. The target nucleic acids may be DNA, RNA, and/or variants thereof. Nucleic acids of interest can be isolated and purified from the sample sources before applying methods of the present invention. Preferably, the target nucleic acids are sufficiently free of proteins and any other substances interfering with primer-extension and/or detection reactions. Many methods, for example, described in Ausubel F. M et al, eds., 1993; Walsh P. S. et al, 1991; Boom W. R. et al, 1993; Miller S. A. et al, 1988, are available for the isolation and purification of nucleic acids of interest including commercial kits and specialty instruments. In a preferred embodiment, the target nucleic acid is DNA. In another embodiment, the target nucleic is RNA. Prior to applying the methods of the invention, a DNA copy (cDNA) of target RNA can be obtained using an oligonucleotide primer that hybridize to the target RNA and extending of this primer in the presence of a reverse transcriptase and nucleoside 5′-triphosphates (dNTPs). The resulting DNA/RNA heteroduplex can then be rendered single-stranded using techniques known in the art, for example, denaturation at elevated temperatures. Alternatively, the RNA strand may be degraded in presence of RNase H nuclease. When the target nucleic acid is RNA, one of P1 or P2 primers of the invention (e.g. methods of FIGS. 2 and 3) can be used as an RT-primer for a reverse transcriptase to initiate synthesis of a cDNA copy of the target nucleic acid.

In the methods, “amplification” and “amplifying” target nucleic acids, in general, refers to a procedure wherein multiple copies of the nucleic acid of interest are generated in the form of DNA copies. The terms “amplicon” or “amplification product” refer to a primer-extension product or products of amplification that may be a population of polynucleotides, single- or double-stranded, that are replicated from either strand or both, or from one or more nucleic acids of interest. Regardless of the originating polymer incorporating the target nucleic acid strand and the amplicons state, e.g. double- or single-stranded, all amplicons which are usually homologous are treated herein as amplification products of the same target nucleic acid including the products of incomplete extension. In particular aspects, the term “homology” and “homologous” refers to a degree of identity between nucleic acids. There may be partial homology or complete homology.

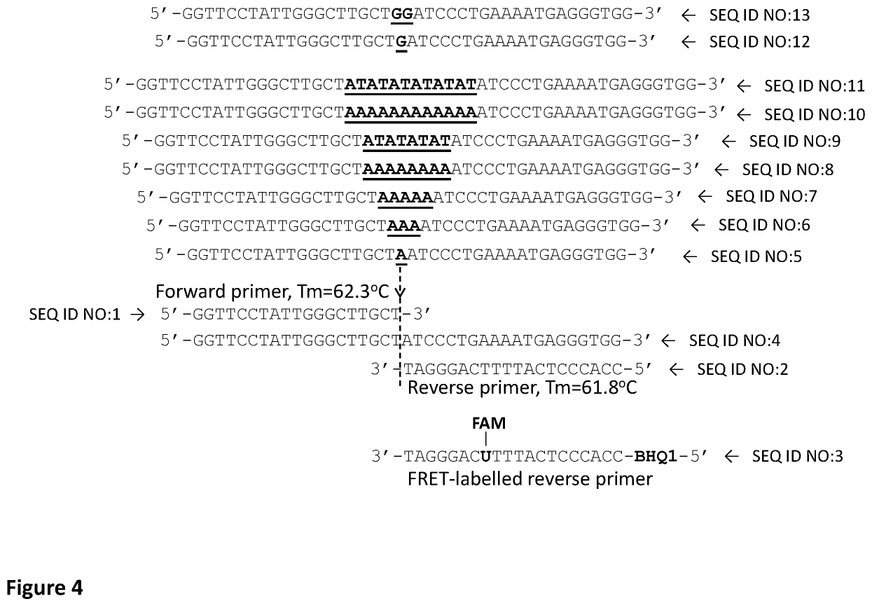

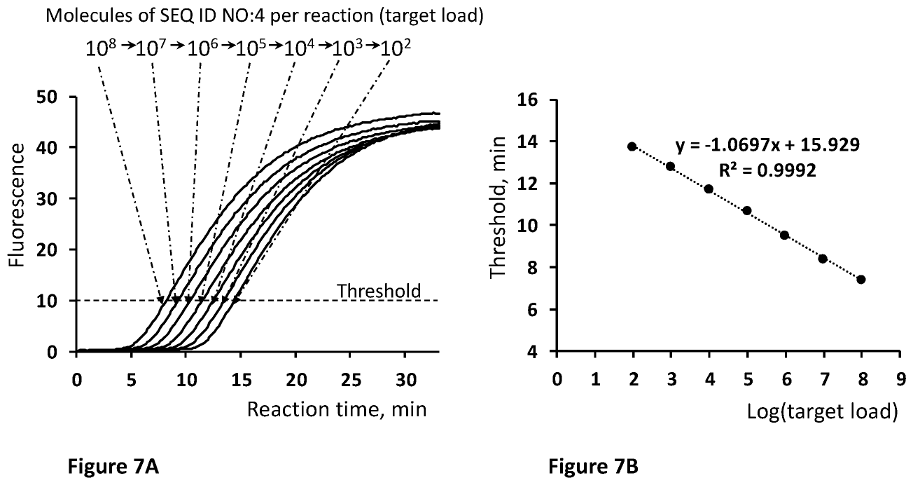

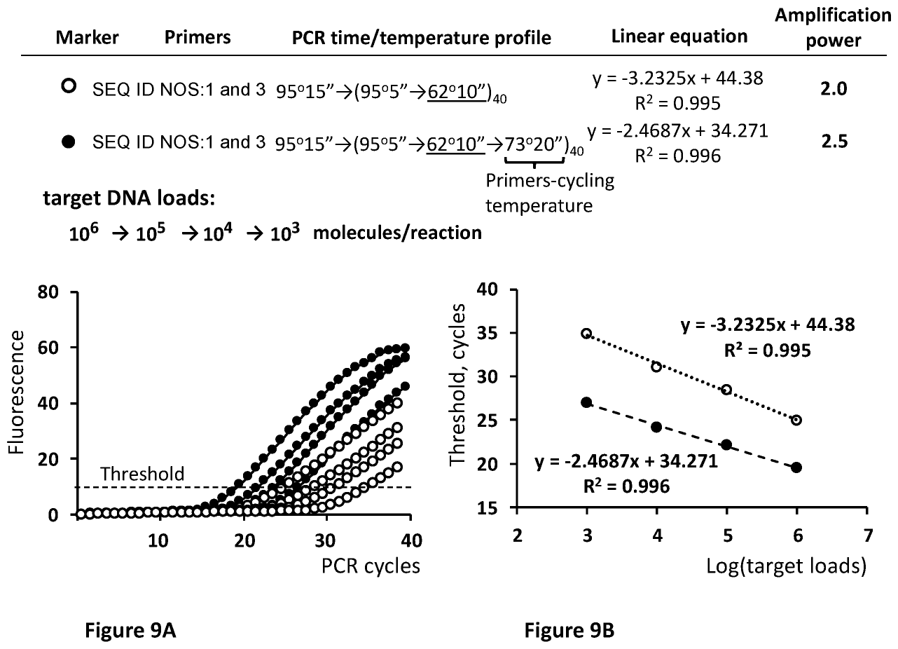

The terms “oligonucleotide primer” and/or “primer” refer to a single-stranded DNA or RNA molecule that hybridizes to a target nucleic acid and primes enzymatic synthesis of a second nucleic acid strand in presence of a DNA polymerase activity. In this case, as used herein, the target nucleic acid “serves as a template” for the oligonucleotide primer. Primers of the invention that perform at a primer-cycling temperature and control the isothermal amplification process can be termed herein as “cycling” primers. The term an “oligonucleotide probe” or “probe” refers to an oligonucleotide component which is used to detect nucleic acids of interest. These terms encompass various derivative forms such as “hybridization-triggered probe,” “fluorescent probe,” “FRET probe,” etc. Oligonucleotides can serve more than one function in PCR, for example, in methods of the invention an oligonucleotide can be a primer that provides for amplification of a target nucleic acid and it also can serve for the real time detection (i.e. usually a function of a “probe”) when it is appropriately labeled by FRET dyes (e.g., SEQ ID NO:3, FIG. 4) as exemplified in FIGS. 7 and 9.

In methods of the invention, “sample” refers to any substance containing or presumed to contain a nucleic acid of interest. The term “sample” thus includes but is not limited to a sample of nucleic acid, cell, organism, tissue, fluid, or substance including but not limited to, for example, blood, plasma, serum, urine, tears, stool, respiratory and genitourinary tracts, saliva, semen, fragments of different organs, tissue, blood cells, samples of in vitro cell cultures, isolates from natural sources such as drinking water, microbial specimens, and objects or specimens that have been suspected to contain nucleic acid molecules.

In methods of the invention, the term “reaction mixture” generally means an aqueous solution comprising all the necessary reactants including oligonucleotide components, enzymes, nucleoside triphosphates (dNTPs), ions like magnesium and other reaction components for performing an amplification or detection reaction of the invention or both. Magnesium ion is preferably present in the reaction mixture because it enables catalytic activity of DNA polymerases. Additional, non-necessary components may be included in the reaction mixture, as long as they don't preclude the methods.

In methods of the invention, “polynucleotide” and “oligonucleotide” are used herein interchangeably and each means a linear polymer of nucleotide monomers. Polynucleotides typically range in size from a few monomeric units, e.g. 5-40, when they are usually referred to as “oligonucleotides,” to several thousand monomeric units. The exact size will depend on many factors, which in turn depends on the ultimate function or use of the oligonucleotide. The oligonucleotides may be generated in any manner, including chemical synthesis, DNA replication, reverse transcription, or a combination thereof. Whenever a polynucleotide or oligonucleotide is represented by a sequence of letters, for example, “CCGTATG,” it is understood herein, unless otherwise specified in the text, that the nucleotides are in 5′ to 3′ forward order from left to right and that “A” denotes deoxyadenosine, “C” denotes deoxycytidine, “G” denotes deoxyguanosine, and “T” denotes deoxythymidine. Usually DNA polynucleotides comprise these four deoxyribonucleosides linked by phosphodiester linkage whereas RNA comprises uridine (“U”) in place of “T” for the ribose counterparts.

“Hybridizing,” “hybridization” or “annealing” refers to a process of interaction between two or more oligo- and polynucleotides forming a complementary complex through base pairing which is most commonly a duplex. The stability of a nucleic acid duplex is measured by its melting temperature. “Melting temperature” or “Tm” means the temperature at which a complementary duplex of nucleic acids, usually double-stranded, becomes half dissociated into single strands. These terms are also used in describing stabilities of secondary structures wherein two or more fragments or portions of the same polynucleotide interact in a complementary fashion with each other forming duplexes (e.g., hairpin-like structures). “Hybridization properties” of a polynucleotide means an ability of this polynucleotide or a fragment or portion thereof to form a sequence specific duplex with another complementary polynucleotide or a fragment or portion thereof. The term “hybridization properties” is also used herein as a general term in describing a complementary duplex stability. In this aspect, “hybridization properties” are similar in use to “melting temperature” or “Tm.” “Improved” or “enhanced hybridization properties” of a polynucleotide refers to an increase in stability of a duplex of this polynucleotide with its complementary sequence due to any means including but not limited to a change in reaction conditions such as pH, salt concentration and composition, for example, an increase in magnesium ion concentration, presence of duplex stabilizing agents such as intercalators or minor groove binders, etc., conjugated or not. The hybridization properties of a polynucleotide or oligonucleotide can also be altered by an increase or decrease in polynucleotide or oligonucleotide length. The cause of the hybridization property enhancement or detraction is generally defined herein in context. A simple estimate of the Tm value can be made using the base pair thermodynamics of a “nearest-neighbors” approach (Breslauer K. J. et al, 1986; SantaLucia J. Jr., 1998). Commercial programs, including Oligo™, Primer Design and programs available on the internet like Primer3™ and Oligo Calculator™, may be also used to calculate a Tm of a nucleic acid sequence useful according to the invention. Commercial programs, e.g., Visual OMP™ (DNA software), Beacon designer 7.00™ (Premier Biosoft International), may also be helpful.

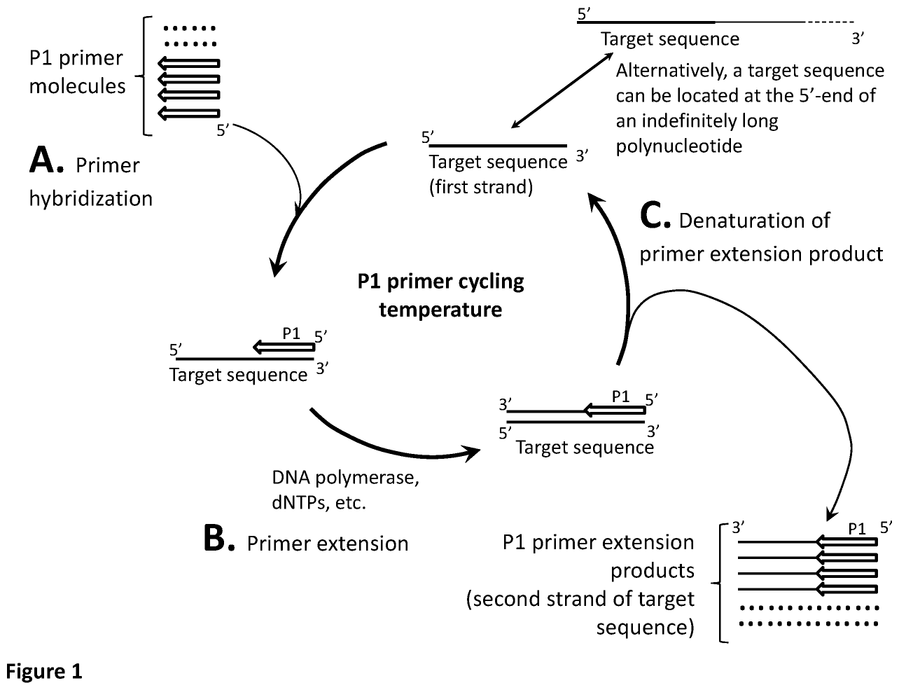

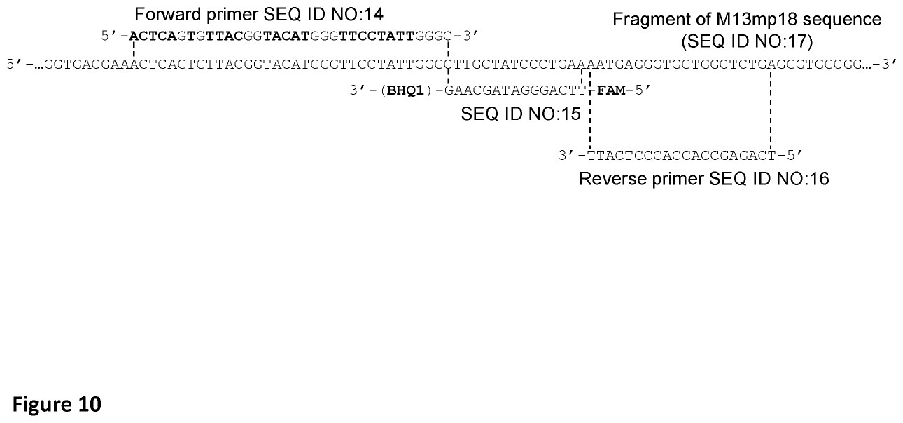

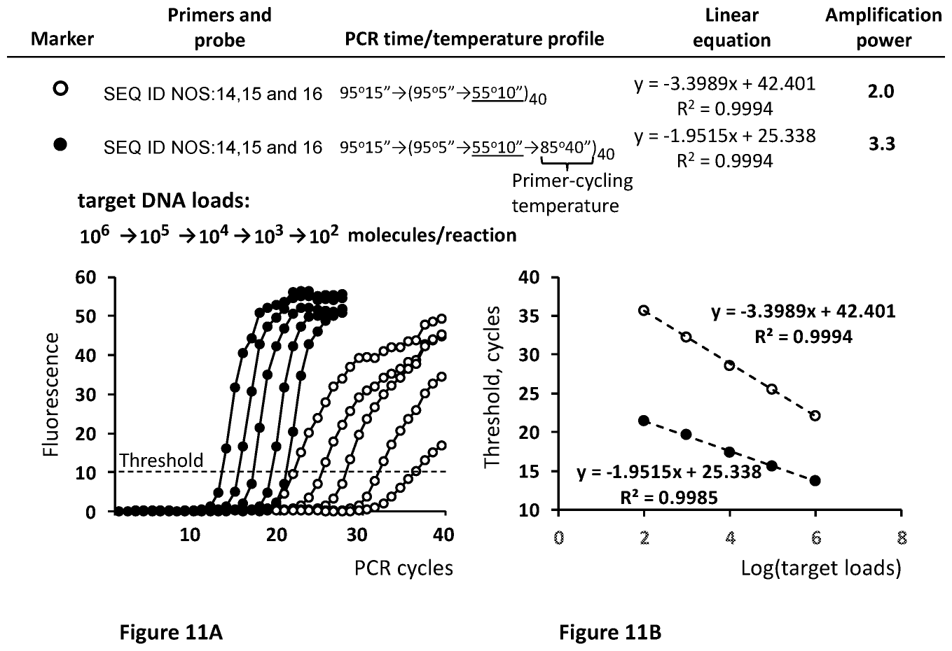

In methods of the invention, the term “structural modifications” refers to any chemical substances such as atoms, moieties, residues, polymers, linkers or nucleotide analogs that are usually of a synthetic nature, and which are not commonly present in natural nucleic acids. “Duplex-stabilizing modifications” refer to structural modifications, the presence of which provide a duplex-stabilizing effect in double-stranded nucleic acids; that is such modifications enhance thermal stability (e.g., “Tm”) relative to nucleic acid duplexes lacking such stabilizing modification(s) (e.g., that contain only natural nucleotides). Conversely, “duplex-destabilizing modifications” refer to structural modifications, the presence of which provide a duplex-destabilizing effect (e.g., decreased thermal stability/Tm) in double-stranded nucleic acids. Duplex-stabilizing modifications include those structural modifications that are most commonly applied in synthesis of probes and primers and are represented by modified nucleotides and “tails” and may include intercalators and minor groove binders. Particularly useful in methods of the invention are “polymerase-compatible” structural modifications incorporated into the oligonucleotide primers. The “polymerase-compatible” structural modifications refer to modifications that do not block DNA polymerase activity in extending the hybridized primers and/or that replicate the primer sequence incorporating these modifications. Use of polymerase-compatible modifications in primer design can be beneficial in methods of the invention. For example, the P1 and P2 primers in methods of FIGS. 1-3 may incorporate polymerase-compatible duplex-stabilizing and/or duplex-destabilizing modifications or a combination thereof. For example, this can be used to adjust the primers' hybridization properties to a particular primer-cycling temperature for more efficient cycling. Another example of polymerase-compatible duplex-stabilizing modification in primer design is shown in FIG. 10 wherein stabilization of the forward primer SEQ ID NO:14 allowed to increase the target amplicon length supporting the FRET-probe detection and efficient cycling of the forward primer at 85° C. as illustrated in FIG. 11. Examples of polymerase-compatible duplex-stabilizing modifications include but are not limited to 5′-conjugated intercalators (e.g. Lokhov, S. G. et al. (1992), minor groove binding moieties (e.g. U.S. Pat. No. 7,794,945 to Hedgpeth J. et al, 2010), 5-methyl cytosine (5-MeC) and 2,6-diamino-purine (2-amA) nucleotide analog in place of cytosine and adenine, respectively (e.g. Lebedev Y. et al, 1996). Examples of polymerase-compatible duplex-destabilizing modifications include but are not limited to 7-deaza purine nucleotide analogs (Seela et al., 1992), deoxyinosine and deoxyuridine nucleotides (Kawase Y. et al, 1986, Martin F. H. et al, 1985).

In the methods, the terms “natural nucleosides” and “natural nucleotides” as used herein refer to four deoxynucleosides or deoxynucleotides respectively which may be commonly found in DNAs isolated from natural sources. Natural nucleosides (nucleotides) are deoxyadenosine, deoxycytidine, deoxyguanosine, and deoxythymidine. The term also encompasses their ribose counterparts, with uridine in place of thymidine. As used herein, the terms “unnatural nucleotides” or “modified nucleotides” refer to nucleotide analogs that are different in their structure from those natural nucleotides for DNA and RNA polymers. Some of the naturally occurring nucleic acids of interest may contain nucleotides that are structurally different from the natural nucleotides defined above, for example, DNAs of eukaryotes may incorporate 5-methyl-cytosine and tRNAs are notorious for harboring many nucleotide analogs. However, as used herein, the terms “unnatural nucleotides” or “modified nucleotides” encompasses these nucleotide modifications even though they can be found in natural sources. For example, ribothymidine and deoxyuridine are treated herein as unnatural nucleosides. In this aspect, the discussed above deoxyinosine and deoxyuridine nucleosides are unnatural nucleosides.

In methods of the invention, the terms “complementary” or “complementarity” are used herein in reference to the polynucleotides base-pairing rules. Double-stranded DNA, for example, consists of base pairs wherein, for example, G forms a three hydrogen bonds, or pairs with C, and A forms a two hydrogen bonds complex, or pairs with T, and it is regarded that G is complementary to C, and A is complementary to T. In this sense, for example, an oligonucleotide 5′-GATTTC-3′ is complementary to the sequence 3′-CTAAAG-5′. Complementarity may be “partial” or “complete.” In partial complementarity, only some of the nucleic acids' bases are matched according to the base pairing rules. The degree of complementarity between nucleic acid strands has significant effects on the strength of hybridization between nucleic acids. This is particularly important in performing amplification and detection reactions that depend upon nucleic acid binding interactions. The terms may also be used in reference to individual nucleotides and oligonucleotide sequences within the context of polynucleotides. As used herein, the terms “complementary” or “complementarity” generally refer to the most common type of complementarity in nucleic acids, namely Watson-Crick base pairing as described above, although the primers, probes and amplification products of the invention may also participate, including in intelligent design, in other types of “non-canonical” pairings like Hoogsteen, wobble and G-T mismatch pairing.

“PCR” is an abbreviation of term “polymerase chain reaction,” the art-recognized nucleic acid amplification technology (e.g., U.S. Pat. Nos. 4,683,195 and 4,683,202 to Mullis K. B.). The commonly used conventional PCR protocol employs two oligonucleotide primers, one for each strand, designed such that extension of one primer provides a template for the other primer in the next PCR cycle. Generally, a PCR reaction consists of repetitions (or cycles) of (i) a denaturation step which separates the strands of a double-stranded nucleic acid, followed by (ii) an annealing step, which allows primers to hybridize to positions flanking a sequence of interest, and then (iii) an extension step which extends the primers in a 5′ to 3′ direction, thereby forming a nucleic acid fragment complementary to the target sequence. Each of the above steps may be conducted at a different temperature using an automated thermocycler. The PCR cycles can be repeated as often (as many times) as desired resulting in an exponential accumulation of a target DNA amplicon fragment whose termini are usually defined by the 5′-ends of the primers used. Particular temperatures, incubation times at each step and rates of change between steps (temperature ramping rates) depend on many factors and examples can be found in numerous published protocols (e.g., McPherson M. J. et al., 1991 and 1995). Although conditions of PCR can vary in a broad range, a double-stranded target nucleic acid is usually denatured at a temperature of >90.degree. C., primers are annealed at a temperature in the range of about 50-70.degree. C., and the extension is preferably performed in the 70.degree. C.-74.degree. C. range. The term “PCR” encompasses derivative forms of the reaction, including but not limited to, “RT-PCR,” “real-time PCR,” “asymmetric PCR,” “nested PCR,” “quantitative PCR,” “multiplexed PCR,” and the like. Cycles in PCR are separated from each other by a denaturation temperature or denaturation step at which all double-stranded products of the primers' extensions are melted. DNA amplification in PCR takes place at lower temperatures than denaturation, and it does not matter whether denaturation step is programed to start or end a PCR cycle. Target nucleic acid can be a fragment or contiguous portion of a very long double-stranded molecule, and therefore, prior to PCR cycling, the reaction protocols commonly incorporate an incubation at a denaturation temperature or greater for a sufficient time to render the polymer single stranded. The denaturation temperature does not need to be kept constant through all cycles of PCR. For example, after few initial cycles of PCR with accumulation of amplification products defined by the sequences of primers used, the denaturation temperature can be lowered such as only these products denature while the primer extension products with indefinite 3′-ends remain double-stranded. However, this is not recommended because this excludes the primer extension products with indefinite 3′-ends from the amplification process and can reduce the overall PCR amplification power including in the accelerated PCR methods of the invention described herein (see FIGS. 9 and 11).

In conventional PCR, the number of amplification products comprising target nucleic acid sequence can double in each consecutive cycle, if quantitative yield is achieved in primer annealing and extension reactions. Then the number or concentration (C) of target nucleic acid sequence in each PCR cycle can be calculated using a simple equation C=2ⁿ×C₀ wherein ‘n’ is the cycle number and ‘C₀’ is the initial target load in a sample or reaction. The term “target load” means initial concentration or number of molecules or “copies” of target nucleic acid sequences in a sample or PCR reaction.

As used herein in the methods, the term “accelerated PCR” means a PCR method wherein the number of amplification products or molecules comprising target nucleic acid sequence can more than double in one or more, a plurality of, many, most, a majority of consecutive cycles. Similar to conventional PCR, in methods of the invention the number or concentration (C) of target nucleic acid sequences and target amplification products in each PCR cycle can be calculated using an exponential equation C=bⁿ×C₀ wherein ‘n’ is the cycle number, ‘C₀’ is the initial target load and ‘b’ is a base number that is, in methods of the invention greater than 2 and that is commonly referred to herein as “amplification power” or “amplification power coefficient.” The amplification power coefficient can be determined by a method that is well established in the art and that is based on target load titration as illustrated herein in FIGS. 9 and 11. The amplification power coefficient ‘b’ was calculated according to equation b=10^{(1/-s)} using the slope values (“s” in the equation) from the linear trend equation (see FIGS. 9 and 11). In this aspect, the amplification power coefficients determined herein represent an average value throughout/over most or all PCR cycles.

The term “isothermally-accelerated amplification” collectively relates herein to the methods of the invention, wherein one of two (FIGS. 1 and 3), or both (FIG. 2), forward and reverse primers hybridize to the corresponding target strand, become extended by a DNA polymerase, and wherein their respective extension product(s) denature at the same “cycling” temperature (i.e., isothermally). These primers (e.g., P1 and P2) can be referred to herein as “forward” and “reverse.” This forward/reverse terminology, however, does not necessarily assign any special properties to the primers other than their relation to each other, the target nucleic acid sequence, and the target strands.

In the methods, the phrase “incubating the reaction mixture at a primer-cycling temperature,” as used herein, means an exposure of the reaction mixture to a temperature or temperature range that supports all three steps of the isothermal amplification reaction of the invention, i.e., (i) hybridization of a primer to a target template strand, (ii) extension of the primer by a DNA polymerase to produce a double-stranded target amplicon, and (iii) denaturation of the amplicon providing two target strands single-stranded, one of which serves as a primer template for another molecule of the primer in the next consecutive isothermal cycle (e.g. FIG. 1). In this aspect, with respect to each primer, the term “isothermal cycle” means completion of these reaction steps. Incubation of the reaction mixture at a primer-cycling temperature leads to repetition of the isothermal cycles or “isothermal cycling” resulting in the target amplification. In preferred aspects the primer-cycling temperature for both primers bracketing a target sequence is the same, or substantially the same. In the methods of the invention, the reaction mixture may be incubated at a particular primer-cycling temperature at which maximal acceleration of the amplification, i.e. reaction productivity or speed is achieved. This temperature, that can be a narrow temperature range, can be termed herein as an “optimal primer-cycling temperature.” The amplification reaction slows down at the reaction temperatures that are above or below the optimal primer-cycling temperature or temperature range. Alternatively, in the methods, the temperature may change or fluctuate during the incubation between the practically-useful lower and upper limits of the primer-cycling temperature range. Accelerated amplification of the invention is usually triggered by a polynucleotide comprising a target sequence, preferably at the 5′-end of the polymer, and the entire amplification can be performed isothermally (e.g. FIGS. 1,2, 5-7). In other aspects, a reaction temperature change is required during the amplification process (e.g. FIGS. 3, 9 and 11). In these methods, incubation at a primer-cycling temperature may be an important amplification-accelerating period of a more intricate/complex reaction temperature regime. The longer the reaction mixture is incubated at the primer-cycling temperature, the greater the amount of a target sequence can be amplified. In some embodiments, for example, when methods of the invention are applied as part of PCR (FIGS. 9 and 11), the primer-cycling temperature or temperature range may be selected so that the desired amplification effect can be reached at a short time like, e.g. one or few seconds. Depending on the primer-cycling temperature or range, the time of exposure of the reaction mixture at the primer-cycling temperature may be, for example, 1 second or longer, preferably 2, 3, 4 seconds or longer and more preferably 5, 6, 7, 8, 9 or 10 seconds or longer, or 30 seconds or longer. The longer the time of exposure of the reaction mixture at the primer-cycling temperature or temperature range, the greater the level of target amplification that can be achieved, and the greater the amplification power of the PCR. Regarding the PCR implementations, optimization of the primer-cycling temperature and incubation time at the primer-cycling temperature are preferably selected to increase the amplification power of PCR to a value greater than 2, greater than 2.5, or preferably greater than 5, etc., to provide the greatest accelerated PCR.

In methods of the invention, the term “design” in the context of the method steps and/or oligonucleotides, etc., has broad meaning, and in certain aspects is equivalent to the term “selection.” For example, the terms “oligonucleotide design,” “primer design,” “probe design” can mean or encompass selection of a type, a class, or one or more particular oligonucleotide structure(s) including the nucleotide sequence and/or structural modifications (e.g., labels, modified nucleotides, linkers, etc.). The term “system design” generally incorporates the terms “oligonucleotide design”, “primer design”, “probe design” and also refers to relative orientation and/or location of the oligonucleotide components and/or their binding sites within the target nucleic acids. In these aspects, the term “assay design” relates to the selection of any, sometimes not necessarily to a particular, methods including all reaction conditions (e.g. temperature, salt, pH, enzymes, oligonucleotide component concentrations, etc.), structural parameters (e.g. length and position of primers and probes, design of specialty sequences, etc.) and assay derivative forms (e.g. post-amplification, real-time, immobilized, FRET detection schemes, etc.) chosen to amplify and/or to detect the nucleic acids of interest.

In methods of the invention, “detection assay” or “assay” refers a reaction or chain of reactions that are performed to detect nucleic acids of interest. The assay may comprise multiple stages including amplification and detection reactions performed consecutively or in real-time, nucleic acid isolation and intermediate purification stages, immobilization, labeling, etc. The terms “detection assay” or “assay” encompass a variety of derivative forms of the methods of the invention, including but not limited to, a “post-amplification assay” when the detection is performed after the amplification stage, a “real-time assay” when the amplification and detection are performed simultaneously, a “FRET assay” when the detection is based using a FRET effect, “immobilized assay” when one of either amplification or detection oligonucleotide components or an amplification product is immobilized on solid support, and the like.

Methods of the invention can be used to amplify and detect one, or a plurality (more than one) of target nucleic acids in, for example, a multiplex detection format. The term “multiplexed detection” refers to a detection reaction wherein multiple or plurality of target nucleic acids are simultaneously detected. “Multiplexed amplification” correspondingly refers to an amplification reaction wherein multiple target nucleic acids are simultaneously amplified in the same reaction mixture.

In methods of the invention, products of the target amplification can be detected by any appropriate physical, chemical or biochemical approach. In preferred embodiments, the amplification products comprise a detectable label. The term “label” refers to any atom or molecule that can be used to provide a detectable signal and that can be attached to a nucleic acid or oligonucleotide. Labels include but are not limited to isotopes, radiolabels such as ³²P, binding moieties such as biotin, haptens, mass tags, phosphorescent or fluorescent moieties, fluorescent dyes alone or in combination with other dyes or moieties that can suppress or shift emission spectra by FRET effects. Labels may provide signals detectable by fluorescence, radioactivity, colorimetry, gravimetry, X-ray diffraction or absorption, magnetism, enzymatic activity, mass spectrometry, binding affinity and the like. A label may be a charged moiety or alternatively, may be charge neutral. Labels can include or consist of nucleic acid or protein sequences, so long as the sequence comprising the label is detectable. In preferred embodiments, the label is a fluorescent label. “Fluorescent label” refers to a label that provides a fluorescent signal. A fluorescent label is commonly a fluorescent dye, but it may be any molecule including but not limited to a macromolecule like a protein, a particle made from inorganic material like quantum dots, as described, for example, in (Robelek R. et al, 2004), etc.

In methods of the invention, the probes may be FRET probes and the detection of target nucleic acids may be based on FRET effects. “FRET” is an abbreviation of Forster Resonance Energy Transfer effect. FRET is a distance-dependent interaction occurring between two dye molecules in which excitation is transferred from a donor to an acceptor fluorophore through dipole-dipole interaction without the emission of a photon. As a result, the donor molecule fluorescence is quenched, and the acceptor molecule becomes excited. The efficiency of FRET depends on spectral properties, relative orientation and distance between the donor and acceptor chromophores (Forster T., 1965). As used herein, “FRET probe” or “FRET primer” refers to a fluorescent oligonucleotide that is used for detection of a nucleic acid of interest, wherein detection is based on FRET effects. The acceptor chromophore may be a non-fluorescent dye chosen to quench fluorescence of the reporting fluorophore (Eftink M. R., 1991). Formation of sequence-specific hybrids between the target nucleic acid and the probes or primer leads to changes in fluorescent properties providing for detection of the nucleic acid target. FRET is widely used in biomedical research and particularly in probe designs for nucleic acid detection (e.g., in Didenko V. V., 2001).

Many detection strategies and designs exploiting the FRET effect are known in the art, and these strategies may be used in design of the FRET-labeled probes or FRET-labeled primers of the invention. In particular aspects, the FRET probes or FRET primers are hybridization-triggered FRET oligonucleotide components. The “hybridization-triggered” FRET approach is based on distance change between the donor and acceptor dyes as result of a sequence specific duplex formation between a target nucleic acid and a fluorescent oligonucleotide component. When a FRET-labeled oligonucleotide component is unhybridized, the quencher moiety is sufficiently close to the reporter dye to quench its fluorescence due to random oligonucleotide coiling. Once the FRET-labeled oligonucleotide component is hybridized to the primer-extension products forming rigid duplex, the quencher and reporter moieties are separated, thus enabling the reporter dye to fluoresce providing for the target nucleic acid detection (e.g., Livak K. J. et al, 1998). Examples of other hybridization-triggered FRET system designs that can be used practicing the present invention include but not limited to “Adjacent Hybridization Probe” method (e.g. Eftink M. R., 1991; Heller M. J. and Morrison L. E., 1985), “Molecular Beacons” (Lizardi P. M. et al, 1992), “Eclipse Probes” (Afonina I. A. et al, 2002), all of which are incorporated herein by reference for their relevant teachings. The exemplary experimental results shown of FIGS. 7 and 9 and discussed in working Examples 3.3 and 3.5 illustrate an embodiment of the invention wherein a FRET-labeled reverse primer (SEQ ID NO:3, FIG. 4) was used for the detection of amplified material. Other approaches that are based on use of FRET-labeled primers and that can be used in methods of the invention include those described, for example, in U.S. Pat. No. 9,353,405 to Rabbani E. et al (2016); U.S. Pat. No. 5,866,336 to Nazarenko I. A. et al (1999); U.S. Patent Application Publ. No. 2012/0058481 to Ge W. et al, all of which are incorporated herein by the reference.

In methods of the invention, the amplification and detection stages of the invention may be performed separately when the detection stage follows the amplification. The terms “detection performed after the amplification,” “target nucleic acid is amplified before the detection reaction” and “post-amplification detection” are used herein to describe such assays. In preferred method embodiments of the invention, detection of target nucleic acids can be performed in “real-time.” Real-time detection is possible when all detection components are available during the amplification, and the reaction conditions (e.g., temperature, buffering agents to maintain pH at a selected level, salts, co-factors, scavengers, and the like) support both amplification and detection stages of the reaction. This permits a target nucleic acid to be measured as the amplification reaction progresses, decreasing the number of subsequent handling steps required for the detection of amplified material. “Real-time detection” means an amplification reaction for which the amount of reaction product, (e.g., target nucleic acid sequences), is monitored as the reaction proceeds. Reviews of the detection chemistries for real-time amplification can be found, for example, in Didenko V. V. (2001); Mackay I. M. et al (2002), and Mackay J., Landt O. (2007), which are incorporated herein by reference for their relevant teachings. In preferred embodiments of the present invention, real-time detection of nucleic acids is based on use of FRET effect, FRET-labeled probes or primers. In certain aspects, detection of amplified nucleic acid material can be performed using certain technologies based on nuclease-cleavable probes. Examples include but are not limited to chimeric DNA-RNA probes that are cleaved by RNAse H upon the binding to target DNA (see, e.g. U.S. Pat. No. 4,876,187 to Duck P. et al, 1989); target-specific probe cleavage based on the substrate specificity of Endonuclease IV and Endonuclease V from E. coli (PCT Patent Application WO/2007/127999 and PCT Patent Application WO/2007/127992 to Kutyavin I. V.); methods enhancing 5′-nuclease cleavable FRET-probes (U.S. Pat. No. 9,121,056 (2015) and U.S. Pat. No. 9,914,963 (2018) to Kutyavin I. V.).

The reaction components to perform methods of the invention can be delivered in the form of a kit. As used herein, the term “kit” refers to any system for delivering materials. In the context of methods/reaction assays, such delivery systems include elements allowing the storage, transport, or delivery of reaction components such as oligonucleotides, buffering components, additives, reaction enhancers, enzymes and the like in the appropriate containers from one location to another commonly provided with written instructions for performing the assay. Kits may include one or more enclosures or boxes containing the relevant reaction reagents and supporting materials. The kit may comprise two or more separate containers wherein each of those containers includes a portion of the total kit components. The containers may be delivered to the intended recipient together or separately.

The oligonucleotide components of the invention such as primers and probes can be prepared by a suitable chemical synthesis method. The preferred approach is the diethylphosphoramidate method disclosed in Beaucage S. L., Caruthers M. H. (1981), in combination with the solid support method disclosed in U.S. Pat. No. 4,458,066 to Caruthers M. H., Matteucci M. D. (1984) and performed using one of commercial automated oligonucleotide synthesizer. When oligonucleotide components of the invention, primers or probes, need to be labeled with a fluorescent dye a wide range of fluorophores may be applied in designs and synthesis. Available fluorophores include but not limited to coumarin, fluorescein (FAM, usually 6-fluorescein or 6-FAM), tetrachlorofluorescein (TET), hexachloro fluorescein (HEX), rhodamine, tetramethyl rhodamine, BODIPY, Cy3, Cy5, Cy7, Texas red and ROX. Fluorophores may be chosen to absorb and emit in the visible spectrum or outside the visible spectrum, such as in the ultraviolet or infrared ranges. FRET probes or primers of the invention commonly incorporate a pair of fluorophores, one of which may be a none-fluorescent chromophore (commonly referred as a “dark quencher”). Suitable dark quenchers described in the art include Dabcyl and its derivatives like Methyl Red. Commercial non-fluorescent quenchers, e.g., Eclipse™ (Glen Research) and BHQ1, BHQ2, BHQ3 (Biosearch Technologies), may be also used for synthesis of FRET primers and probes of the invention. Preferred quenchers are either dark quenchers or fluorophores that do not fluoresce in the chosen detection range of the assays. Modified nucleoside or nucleotide analogs, for example, 5-methyl cytosine, 2-amino adenosine (2,6-diaminopurine), deoxyinosine and deoxyuridine, which are rarely present in natural nucleic acids may be incorporated synthetically into oligonucleotide components. The same applies to linkers, spacers, specialty tails like intercalators and minor groove binders. All these chemical components can be prepared according to methods of organic chemistry or using respective protocols that can be found in manuscripts and patents cited herein. Many structural modifications and modified nucleosides useful to prepare oligonucleotide components of the invention are available, commonly in convenient forms of phosphoramidites and specialty-controlled pore glass, from commercial sources, e.g., Glen Research, Biosearch Technologies, etc.

DNA polymerases are key components in practicing amplification and detection assays of the invention. DNA polymerases useful according to the invention may be native polymerases as well as polymerase mutants, which are commonly modified to improve certain performance characteristics or to eliminate 5′ to 3′ and/or 3′ to 5′ exo nuclease or endo nuclease activities that may be found in many native enzymes. Nucleic acid polymerases can possess different degrees of thermostability. Preferably, for performing the PCR methods of the invention, DNA polymerases are stable at temperatures >90° C., preferably >95° C., and even more preferably >100° C. Examples of thermostable DNA polymerases which are useful for performing the PCR methods of the invention include but are not limited to Vent™, Vent(exo-)™, Deep Vent™, Deep Vent(exo-)™ (New England Biolabs), SD polymerase (Bioron GmbH), Top polymerase (Bioneer), Taq DNA polymerase and other polymerase from Thermus species. The presence or absence in DNA polymerases of the 3′ to 5′ nuclease activity, which is known in the art as “proofreading” nuclease activity, is not as significant for many methods of the invention as other characteristics such as the enzyme processivity, affinity to the primer-extension complex, and DNA synthesis speed.

### Exemplary Factors and Conditions for Thermokinetically Balanced Isothermal Amplification of Nucleic Acid Sequences

### Thermodynamic Balancing of Hybridization Properties (Tm's) of Primers and Respective Target Amplicon in Methods of the Invention.

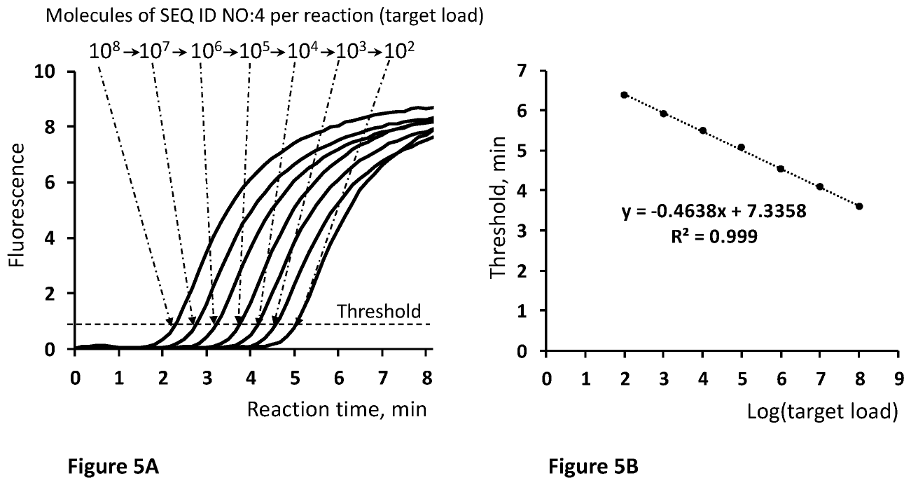

For practicing of methods of FIG. 2, when both P1 and P2 primers are ‘cycling’ at the same primer-cycling temperature, it is preferred to select the primer sequences such that they have hybridization property (melting temperatures or Tm's) that are as close to the same as possible. Disbalancing the primers in their relative thermal stability may slow down the isothermal amplification process. An example, using the reaction scheme of FIG. 2, of a very fast (<6 mins) isothermal target amplification (at a primer-cycling temperature of 74° C.) using a pair of well-balanced primers (SEQ ID NOS:1 and 2, FIG. 4) is shown in FIG. 5. As shown in FIG. 7, however, FRET-labeling of one of these primers (SEQ ID NO:3, FIG. 4) was suspected of affecting, likely negatively, on the balanced primer hybridization property and thus disbalanced the primer-cycling mechanism. Accordingly, the optimal primer-cycling temperature for the FRET-labeled primer was reduced by one degree (to 73° C., which was the reaction temperature used to generate the data of FIG. 7) resulting in an amplification rate (FIG. 7) that was approximately 2-times slower than that of FIG. 5. According to further aspects, use of DNA polymerase-compatible duplex-stabilizing or destabilizing modifications, selection of the primers' length and composition, or a combination thereof in primer design may be used to tune up and therefore balance their hybridization properties for best performance. Alternatively, one or both primers may incorporate an NT-rich sequence at 5′-end that is not complementary to the target nucleic acid and that is appropriately labeled with FRET dyes. The length and composition of this FRET-labeled 5′-sequence can be selected such as it provides very little, if any effect on the original primers' hybridization properties (primers without the ‘artificial’ 5′-FRET-sequence) as well as stability of the corresponding reaction amplicon.

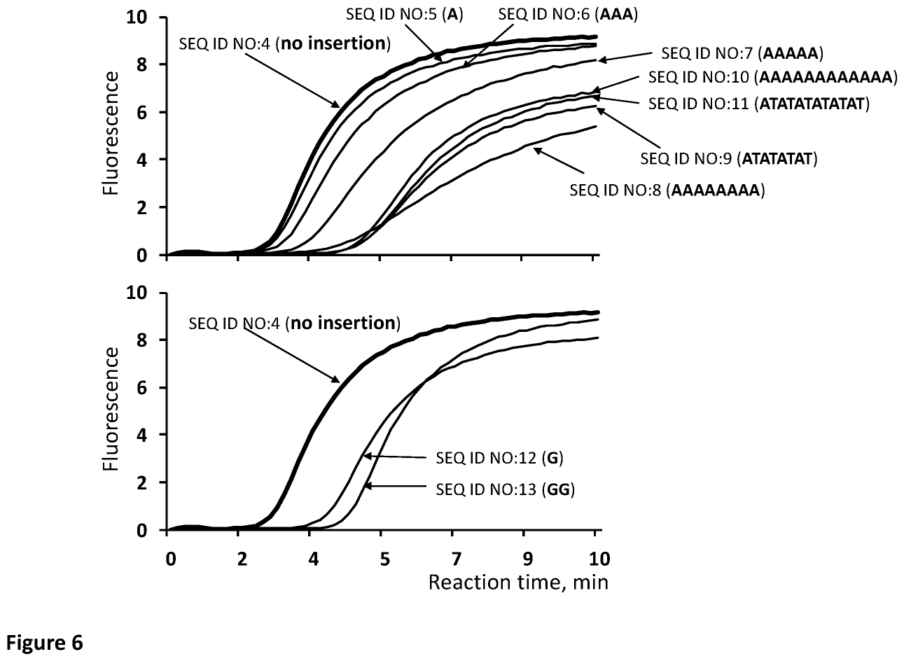

Practicing the methods of the invention place certain limits on the length of the target amplicons and their hybridization properties. For example, to be practically useful, the extended target sequence primed by the P1 primer in methods of FIG. 1 must be long enough to incorporate a P2 primer binding site. The optimal length of the extended portion of the primer-extension product depends on its nucleotide composition, but in any case, the extended portion (not counting the primer) is preferably longer than 8, longer than 10, longer than 12 or longer than 15 nucleotides, especially in the cases of mixed NT and G/C-comprising extended portion sequences. For example, in the experiment of FIG. 11, the extended portion of the primer-extension product of cycling primer SEQ ID NO:14 was 34-mer long and comprised a mixed A/T/G/C-sequence (see FIG. 10). Methods according to the scheme of FIG. 2 are also restricted with respect to the length of the target amplicons. The distance, in nucleotides, between the 5′ end of first primer binding site on the first strand and the 5′ end of the second primer binding site on the second strand within the target sequence template strands is less than 20, less than 15, less than 10, less than 5, less than 4, less than 3, less than 2, 1, or 0 (or is a value in the range of 0-20, or in any subrange thereof) as this was illustrated by the target sequences (SEQ ID NO: 4-13) and primers (SEQ ID NO: 1 and 2; FIG. 4) and experiments of FIG. 6. Ideally, the length of the target amplicons is equal to the sum of nucleotides in both P1 and P2 primers. The presence of target sequence nucleotides between the primers or their respective binding site sequences slows down the isothermal amplification as illustrated by the experiment of FIG. 6. The amplification reaction may tolerate one or a few A/T-bases ‘target insertions’ with a minor negative effect on the reaction rate, but this is not the case for G/C-insertions, which typically substantially slow the reaction rate. Unlike the methods of FIG. 2, the methods of FIG. 1 as well as the methods of FIG. 3, that is an extension of the methods of FIG. 1, are less restricted regarding the expansion of the target amplicon length. As illustrated in FIG. 11, improvement of the hybridization property of the P1 ‘cycling’ primer by use of the DNA polymerase-compatible duplex-stabilizing nucleotide modifications in its design can be particularly advantageous. This allowed for increasing the target amplicon length and adoption of a FRET-probe detection (see FIG. 10) scheme, while maintaining good primer-cycling performance at 85° C. with respect to the P1 primer.

### Primer-Cycling Temperature Optimization.

In thermokinetically-balanced methods of the invention all three steps of primer hybridization, polymerase-assisted primer extension and denaturation of the target duplex are taking place at the same temperature (isothermally). Sufficient denaturation of the target duplex to render that strands primable is a critical step of the isothermal amplification. According to particular aspects, the primer-cycling temperature may be found at or near the temperature at which the target amplicon begins to melt or initiates melting. This, in turn, defines a temperature range for determination of the optimal primer-cycling temperature that can be found experimentally for any cycling primer (FIGS. 1 and 3) or a pair of cycling primers (FIG. 2). Determining an optimal temperature or temperature range, that is typically narrow, is important in achieving the maximal amplification rate.

### Thermokinetic Balancing to Accelerate Isothermal Amplification of the Invention.

In methods of the invention, primers, and particularly the cycling primers commonly have weaker hybridization properties than corresponding target sequences (Tm's of the primers' duplexes with the target sequences vs. target sequences duplex Tm). According to additional aspects, however, the ability of the cycling primers to hybridize to the target template strands at a given primer-cycling temperature can be improved kinetically by raising the respective primer concentration in the reaction mixture. Preferably, the cycling primer concentration (nanomolar) in the isothermal cycling reaction mixture should be greater than or equal to 100, greater than or equal to 150, greater than or equal to 200, preferably greater than or equal to 500 and even more preferably greater than or equal to 1000 nanomolar. The greater the concentration of the cycling primers, the greater the rate of isothermal amplification can be reached. The thermokinetically balanced isothermal amplification reactions of the invention thus preferably involve balancing the primer concentrations with the thermal properties of the primers and target sequence.

### Detection of Amplified Target Sequences in Methods of Thermokinetically Balanced Isothermal Amplification.

In preferred aspects, the amplified material of the thermokinetically balanced isothermal amplification is detected in real time using fluorescent label that can be a dye that fluoresces upon binding to target duplex (e.g. see FIGS. 5, 6, and 8), FRET-labeled primer (e.g. see FIGS. 7 and 9), or FRET-labeled probe (e.g. see FIG. 11). Unlike in the fluorescent dye case, use of the FRET-labeled primers or probes enables multiplex amplification and detection of more than one target sequence in the same reaction mixture. Application of the probe approach is preferred in the art because it warrants sequence specificity of the target detection.

### DNA Polymerases and Other Factors in Methods of Thermokinetically Balanced Isothermal Amplification.

DNA polymerase is yet another important component of the isothermal amplification system effecting thermokinetic balancing of the reaction. DNA polymerases in the methods can have either 5′-to-3′ or 3′-to-5′ nuclease activity. Use of DNA polymerases that have 3′-to-5′ nuclease activity, that is also known in the art as a “proof-reading” activity, may be limited, since the enzyme can quickly digest single-stranded oligonucleotides. Structural modification of the primers and probes, like incorporation of phosphorothioate inter-nucleotide linkage at the 3′-end, may be necessary to protect these oligonucleotide components. Alternatively, magnesium salt concentration needs to be adjusted to a lower level of ˜2 millimolar, although this also negatively effect on the DNA polymerase activity. The DNA polymerase-associated 5′-to-3′ exo(endo) nuclease activity can be useful in the case of 5′-nuclease-cleavable FRET-probe as illustrated in FIG. 11.

DNA polymerases differ in many properties like thermal stability, processivity, polymerization speed, strand-displacement etc. Ideally, in the methods of invention, the DNA polymerase extends the hybridized primer to the end of the target template before it leaves the extension complex. Any premature dissociation of the enzyme from the extension complex can lead to denaturation of the truncated duplex at the primer-cycling temperature before the DNA polymerase comes/associates back to finish the extension. The truncated primer-extension product may not incorporate the reverse primer binding site, or have it sufficiently shortened to prevent this product participation in further amplification process, thus slowing down the overall amplification rate. This means that the strength of association of DNA polymerases with the extension complex can affect on rate of the amplification, and it has to be sufficient to accomplish the primer extension at commonly elevated primer-cycling temperatures (e.g. FIG. 11). Therefore, enzymes are preferably selected from DNA polymerases that have lowest Michaelis constant and/or greatest processivity factor in the reaction conditions including the primer-cycling temperatures in methods of the invention. Examples of such enzymes include, but not limited to Vent(exo-) and Deep Vent(exo-) DNA polymerases.

### Target Sequence and Initiation of Thermokinetically Balanced Isothermal Amplification.

Any single-stranded polynucleotide comprising target sequence can trigger the thermokinetically balanced isothermal amplification. This polynucleotide can be a DNA or RNA. When the polynucleotide is RNA, a DNA copy (cDNA) of target RNA can be obtained using an oligonucleotide primer that hybridize to the target RNA and extending of this primer in the presence of a reverse transcriptase and nucleoside 5′-triphosphates (dNTPs). The resulting DNA/RNA heteroduplex can then be rendered single-stranded using techniques known in the art, for example, denaturation at elevated temperatures. Alternatively, the RNA strand may be degraded in presence of RNase H nuclease. When the target nucleic acid is RNA, one of P1 or P2 primers of the invention (e.g. methods of FIGS. 2 and 3) can be used as an RT-primer for a reverse transcriptase to initiate synthesis of a cDNA copy of the target nucleic acid.

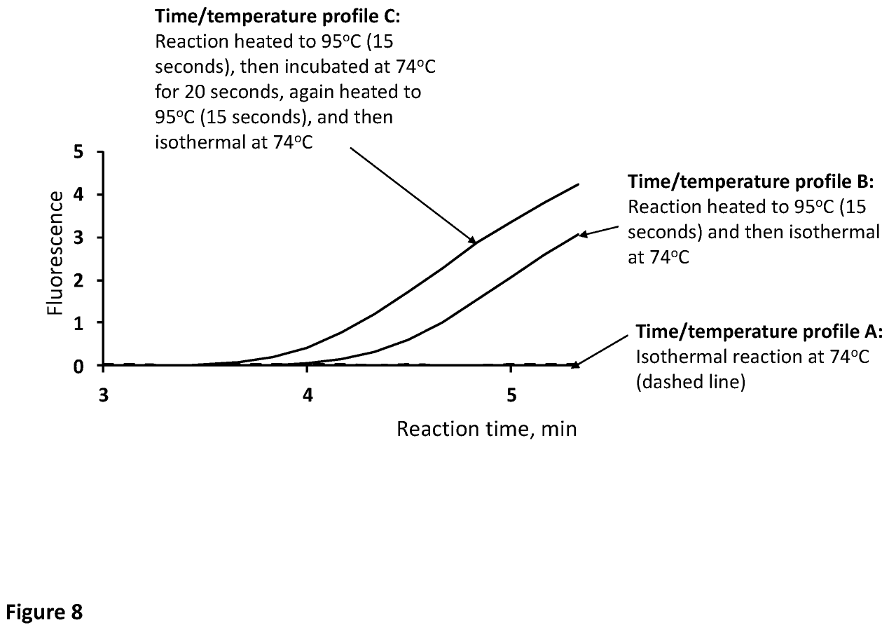

When the polynucleotide is DNA, the most effective amplification-triggering component is either strand of a target sequence or polynucleotide incorporating a target sequence at its 5′-end. Experimental results of FIG. 8 show that a single-stranded DNA polymer incorporating the target sequence somewhere in a middle of the nucleotide chain can also initiate the amplification, although with slight time delay. Results of FIG. 8 also show that a double-stranded DNA incorporating a target sequence cannot trigger the amplification process of the invention unless it is rendered single-stranded.

Combining Thermokinetically Balanced Isothermal Amplification with Other Amplification Methods.

Many methods have been discovered and developed in the art wherein nucleic acid amplification is performed at a steady temperature. Examples of these isothermal amplification technologies include, but not limited to NASBA (e.g., U.S. Pat. No. 6,063,603), HDA (e.g., Vincent M. et al, 2004), Rolling Circle Amplification (e.g., U.S. Pat. Nos. 5,854,033 and 6,210,884 to Lizardi P., 1998 and 2001), Loop-mediated isothermal amplification (e.g., U.S. Pat. No. 6,410,278 to Notomi T. and Hase T., 2002), amplification methods based on the use of RNA or composite RNA/DNA primers (e.g., U.S. Pat. No. 5,824,517 to Cleuziat P. and Mandrand B., 1998; U.S. Pat. No. 6,251,639 to Kurn N., 2001), Strand Displacement Amplification (e.g., U.S. Pat. No. 5,270,184 to Walker G. T. et al, 1993; U.S. Pat. No. 5,648,211 to Fraiser M. S. et al, 1997; U.S. Pat. No. 5,712,124 to Walker G. T., 1998), Nick Displacement Amplification (PCT Patent Application WO 2006/125267 to Millar D. S. et al, 2006; U.S. Patent Application Publication 2003/0138800 to Van Ness J. et al, 2003), Accelerated Cascade Amplification (PCT Patent Application WO/2008/086381 to Nelson J. R. et al, 2008; U.S. Pat. No. 8,143,006 to Kutyavin I. V., 2012) and other techniques. In these methods, the amplified nucleic acid sequences can be present in a single-stranded form (e.g. Rolling Circle Amplification), or at least, during a reasonably long time. The thermokinetically balanced amplification is also an isothermal process, and this brings an opportunity of combing this technology with other art-known methods, if the reaction components and conditions of both methods are compatible. By primer design, the thermokinetically balanced amplification can be adapted to virtually any reaction temperature. On the other hand, the art-know amplification systems commonly have a leverage to be performed within a reasonably broad temperature range. The effect of the technologies' combination can be cooperative when an art-known system amplifies and supplies a target-comprising polynucleotide to the thermokinetically balanced amplification system. Regarding the amplification and detection of the amplified material, the art-known techniques have their own advantages and disadvantages. Particularly the advantages of these methods can not only speed up the overall amplification process, but also benefit specificity, sensitivity, multiplexing capabilities and other parameters of the ‘technology-combination’ assay.

Although PCR is based on temperature ramping commonly within a broad temperature range, results of FIGS. 9 and 11 prove that the thermokinetically balanced amplification of the invention can be effectively combined with PCR to accelerate the overall amplification/detection process. No interference between these two different amplification systems was identified, but the benefit of the combination is obvious (see FIGS. 9 and 11), although in these particular examples, the combined assay has not been optimized.

### Kits for Thermokinetically Balanced Isothermal Amplification of Nucleic Acids.

Compositions and systems of the invention include kits comprising oligonucleotide primers having a system design that is compatible with the isothermal accelerated amplification methods. A kit may include other reaction components like a DNA polymerase and supporting materials including instructions to perform the methods of the invention.

Sequence Listing:

SEQ ID NO: 1 (primer) 5′-GGTTCCTATTGGGCTTGCT-3′
SEQ ID NO: 2 (primer) 5′-CCACCCTCATTTTCAGGGAT-5′
SEQ ID NO: 3 (FRET labeled primer) 5′-(BHQ1)CCACCCTCATTT(FAM-U)CAGGGAT-3′
SEQ ID NO: 4 (fragment of target sequence M13mp18)
5′-GGTTCCTATTGGGCTTGCTATCCCTGAAAATGAGGGTGG-3′
SEQ ID NO: 5 (modified fragment of target sequence M13mp18)
5′-GGTTCCTATTGGGCTTGCTAATCCCTGAAAATGAGGGTGG-3′
SEQ ID NO: 6 (modified fragment of target sequence M13mp18)
5′-GGTTCCTATTGGGCTTGCTAAAATCCCTGAAAATGAGGGTGG-3′
SEQ ID NO: 7 (modified fragment of target sequence M13mp18)
5′-GGTTCCTATTGGGCTTGCTAAAAAATCCCTGAAAATGAGGGTGG-3′
SEQ ID NO: 8 (modified fragment of target sequence M13mp18)
5′-GGTTCCTATTGGGCTTGCTAAAAAAAAATCCCTGAAAATGAGGGTGG-3′
SEQ ID NO: 9 (modified fragment of target sequence M13mp18)
5′-GGTTCCTATTGGGCTTGCTATATATATATCCCTGAAAATGAGGGTGG-3′
SEQ ID NO: 10 (modified fragment of target sequence M13mp18)
5′-GGTTCCTATTGGGCTTGCTAAAAAAAAAAAAATCCCTGAAAATGAGGGTGG-3′
SEQ ID NO: 11 (modified fragment of target sequence M13mp18)
5′-GGTTCCTATTGGGCTTGCTATATATATATATATCCCTGAAAATGAGGGTGG-3′
SEQ ID NO: 12 (modified fragment of target sequence M13mp18)
5′-GGTTCCTATTGGGCTTGCTGATCCCTGAAAATGAGGGTGG-3′
SEQ ID NO: 13 (modified fragment of target sequence M13mp18)
5′-GGTTCCTATTGGGCTTGCTGGATCCCTGAAAATGAGGGTGG-3′
SEQ ID NO: 14 (nucleotide-modified primer)
5′-ACTCAGTGTTACGGTACATGGGTTCCTATTGGGC-3′
SEQ ID NO: 15 (FRET probe) 5′-(FAM)TTCAGGGATAGCAAG(BHQ1)-3′
SEQ ID NO: 16 (primer) 5′-TCAGAGCCACCACCCTCATT-3′
SEQ ID NO: 17 (fragment of target sequence M13mp18)
5′-GGTGACGAAACTCAGTGTTACGGTACATGGGTTCCTATTGGGCTTGCTATCCCT
GAAAATGAGGGTGGTGGCTCTGAGGGTGGCGG-3′

### WORKING EXAMPLES

The following working Examples are provided and disclosed for illustrative purposes to demonstrate certain aspects of the invention for amplification and detection of target nucleic acids and are not intended to limit the scope of the inventive methods and applications.

### Example 1

### Materials and Methods

Synthesis of Oligonucleotide Components. Structures and sequences of an exemplary M13mp18 target sequences SEQ ID NOS:4 and 17 was detected using various primers (SEQ ID NOS: 1, 2, 14 and 16) including a FRET-labeled primer (SEQ ID NO:3) and a 5′-nuclease-clevable FRET probe (SEQ ID NO:15) as shown in FIGS. 4 and 10.

For the FRET-labeled primer (SEQ ID NO:3), a BHQ1 “dark” quencher was incorporated onto the 5′-end, and a 6-fluorescein reporting dye was introduced to the middle of the primer using respective phosphoramidites from Glen Research (Cat. NO: 10-5931 and 10-1056). The 5′-nuclease-clevable FRET probe (SEQ ID NO:15) was synthesized using a phosphoramidite derivative of 6-fluorescein for 5′-end incorporation (Cat. NO: 10-1963) and BHQ1-modified controlled pore glass (Cat. NO: 20-5931). Standard and base-modified phosphoramidites, solid supports and reagents to perform the solid support oligonucleotide synthesis were also purchased from Glen Research. 0.25 M 5-ethylthio-1H-tetrazile solution was used as a coupling agent. Oligonucleotides were synthesized either on ABI394 DNA synthesizer (Applied Biosystems) or MerMaid 6™ DNA synthesizer (BioAutomation Corporation) using protocols recommended by the manufacturers for 0.2 micromole synthesis scales. After the automated synthesis, oligonucleotides were deprotected in aqueous 30% ammonia solution by incubation for 12 hours at 55° C., or 2 hours at 70° C.

Tri-ON oligonucleotides were purified by HPLC on a reverse phase C18 column (LUNA 5 μm, 100 A, 250.times.4.6 mm, Phenomenex Inc.) using a gradient of acetonitrile in 0.1 M triethyl ammonium acetate (pH 8.0) or carbonate (pH 8.5) buffer with flow rate of 1 ml/min. A gradient profile including washing stage 0→14% (10 sec), 14→45% (23 min), 45→90% (10 min), 90→90% (5 min), 90→0% (30 sec), 0→0% (7 min) was applied for purification of all Tri-ON oligonucleotides. The product-containing fractions were dried down in vacuum (SPD 1010 SpeedVac™ TermoSavant) and trityl groups were removed by treatment in 80% aqueous acetic acid at room temperature for 40-60 min. After addition to the detritylation reaction (100 μl) of 20 μl sodium acetate (3 M), the oligonucleotide components were precipitated in alcohol (1.5 ml), centrifuged, washed with alcohol and dried down. Concentration of the oligonucleotide components was determined based on the optical density at 260 nm and the extinction coefficients calculated for individual oligonucleotides using on-line OligoAnalyzer™ 3.0 software provided by Integrated DNA Technologies on the company web-site. Based on the measurements, convenient stock solutions in water were prepared and stored at −20° C. for further use. The purity of all prepared oligonucleotide components was confirmed by analytical 8-20% PAAG electrophoresis, reverse phase HPLC and by spectroscopy on Cary 4000 UV-VIS spectrophotometer equipped with Cary WinUV software, Bio Package 3.0 (Varian, Inc.). Oligodeoxyribonucleotides SEQ ID NOS: 4, 5-13 and 16 (shown in FIGS. 4 and 10) were purchased from Integrated DNA Technologies and dissolved in water amounts recommended by the manufacturer for each oligonucleotide to provide stock solutions of 100 micromolar concentration. The melting temperatures (Tm's) (e.g. see FIG. 4) were calculated for a corresponding full complement duplex (200 nM) in 50 mM NaCl, 3 mM MgCl₂, 1.2 mM dNTPs using OligoAnalyzer™ program (see Integrated DNA Technology website).

### Example 2

### A Circular Double-Stranded Cloning Vector M13mp18 was Linearized

The target nucleic acid used in the exemplary target amplification experiments provided herein was selected from the sequence cloning vector M13mp18, which in its double-stranded form is a covalently closed, circular 7,249-base pair DNA. Circular DNAs are very resistant to denaturation unless they linearized, e.g. by restriction nucleases. A reaction mixture of 50 μl of volume was prepared to contain 1 μg of M13mp18 RF I DNA (New England BioLabs, Cat. NO: N4018S), 20 U of EcoR1 endonuclease (New England BioLabs, Cat. NO: R0101S), 1×NEBuffer (supplied with the enzyme). After one-hour incubation at 37° C., the linearized vector was diluted in 20 mM Tris-HCl (pH8) buffer to prepare appropriate stock solutions with DNA concentrations variable in orders of magnitude scale.

### Example 3

### Exemplary Approaches of Accelerated Target Amplification Comprising the Isothermal Primer-Cycling Methods of the Invention were Performed

The PCR reactions provided herein were prepared on ice by mixing the reagent stock solutions. Unless otherwise indicated, all reaction mixtures of 10 μl of final volume incorporated 50 mM KCl, 3 mM Mg(Cl)₂, 20 mM Tris-HCl (pH8), 300 μM each of four 2′-deoxyribonucleoside 5′-triphosphates (dNTPs: dATP, dTTP, dCTP and dGTP), 0.1 mg/ml Bovine Serum Albumin (New England BioLabs, Cat. NO: B9000S), 0.2 U/μl of EvaGreen™ fluorescent dye, when present (Biotium, Cat. NO:31000). The nature and concentration of other components such as oligonucleotide primers, probes, DNA polymerase(s) used in the reactions and the amplification reaction time/temperature profile are indicated in the corresponding figure descriptions (FIGS. 5-9, 11) and in examples below, describing every particular experiment. Circular double-stranded cloning vector M13mp18, linearized as described in Example 2, was used as a sequence of interest (target sequence) in experiments of FIGS. 8 and 9 and 11. Fluorescence monitoring during PCR was conducted using a Rotor-Gene Q real-time instrument (Qiagen). The fluorescence curves shown in FIGS. 5-9 and 11 are an average of at least four parallel reactions. The data (plotted as fluorescence vs. reaction time or PCR cycle) were transferred into an Excel™ format (Microsoft Corporation). The fluorescent curve threshold value (Ct) was determined as the reaction time or PCR cycle number at which the fluorescence signal of particular curve reaches the level indicated by dashed line (see FIGS. 5, 7, 9, and 11).

Example 3.1. The thermokinetically-balanced isothermal amplification method of the invention, the reaction scheme of which is shown in FIG. 2, can be very fast and highly sensitive amplifying and reliably detecting up to hundred molecules of a target nucleic acid as this is illustrated by experimental results of FIGS. 5A and 5B. FIG. 5A is a ‘target-titration’ experiment when the amount of the target load (SEQ ID NO:4, FIG. 4) was varying by order of magnitude from 100 million to 100 molecules per reaction containing forward and reverse primers (SEQ ID NO:1 and 2), both at 2 micromolar concentration, and 0.15 Units per microliter of Vent(exo-) DNA polymerase (New England BioLabs, Cat. NO: M0257S). Structures of the primer and target oligonucleotides are shown in FIG. 4. The amplified target material was detected by monitoring EvaGreen™ fluorescence every 10 seconds in ‘Green’ channel of the instrument set up at ‘Gain 4’. The fluorescence threshold for each curve of FIG. 5A was determined (at dashed line) and plotted versus the logarithm of the target loads in FIG. 5B. The data points of FIG. 5B display good linear trend with an R² value >0.99. The slope coefficient of the linear equations indicates that the amount of the amplified target material in the reaction mixture is increased by an order of magnitude every ˜27 seconds.

Example 3.2. Experimental results of FIG. 6 demonstrate importance of the P1 and P2 primers' selection (i.e. amplification system design) in method of FIG. 2 to achieve maximal amplification speed. Studied was an isothermal amplification of target oligonucleotides (SEQ ID NOS:5-13) that are different from the original M13mp18 target oligonucleotide (SEQ ID NO:4, see structures in FIG. 4) by insertions of artificial nucleotide sequences of various length and compositions between the forward primer (SEQ ID NO:1) and binding site of the reverse primer (SEQ ID NO:2). The amplification reactions of FIG. 6 contained 10⁸ molecules of each investigated target oligonucleotide and signal-collecting channel of the instrument was set up at ‘Gain 6’; otherwise the experiment of FIG. 6 was identical to that of FIG. 5A in the reaction composition and time/temperature profile. Results of the FIG. 6 experiment lead to several conclusions. Appearance of target amplicon base pairs between the primers' sequences negatively effects on the amplification speed, because this stabilizes the target amplicon making it more difficult to melt at the primers-cycling temperature while having no influence on the primers' hybridization properties. Inhibition of the amplification depends not only on the length of the amplicon base pair insertions, but very strongly on the nature of the inserted base pairs. For example, insertions comprising one (SEQ ID NO:5) or three adenosines (SEQ ID NO:6) have little effect on the reaction rate, whereas one or two guanosines insertions noticeably slowed down the amplification to the extent observed for as long as 8-12-mer A/T insertions (FIG. 6). Regarding the isothermal amplification rate, the ‘no insertion’ system design exemplified by the primers and target oligonucleotide (SEQ ID NOS:1, 2 and 4, FIG. 4) is optimal. Obviously, the reduction of the target amplicon length by allowing the primers' overlap, i.e. when the primers becomes complementary to each other by one or more nucleotides at their 3′-end, can further speed up the amplification, but it is not recommended because this promotes a mis-amplification reaction that is well-known in the art as “primer-dimer” formation.

Example 3.3. Use of fluorescent dyes like EvaGreen™ for detection of the amplified material is not applicable for multiplex assays, when two or more target sequences are amplified and individually detected in the same reaction mixture. FRET-effect solved the problem of target multiplexing in the art, and it is most commonly applied in a form of FRET-probes labeled with two dyes and hybridizing to either strand of the target amplicons between the primers' sequences and their binding sites. As has been discussed in Example 3.2 (above), use of FRET-probes in the isothermal amplification methods of FIG. 2 can be limited due to the restrictions on the length and composition of the target sequences between the primers' sequences and their binding sites. However, one of the pair primers (or both) can be labeled by FRET-dyes as this illustrated by the amplification/detection results of FIGS. 7A and 7B. The target-titration experiment of FIG. 7 is identical to the experiment of FIG. 5 in all aspects, but EvaGreen™ dye was omitted, reverse primer SEQ ID NO:2 was replaced with its FRET-labeled analog SEQ ID NO:3 and reaction temperature was reduced by one degree (73° C.) to achieve maximum amplification speed. The reverse primer modification by FRET dyes slowed down the amplification process approximately twice, but the reaction still reliably amplifies and detects up to 100 molecules of the target oligonucleotide in the reaction mixture.

Example 3.4. The isothermal amplification of FIG. 2 method is effectively initiated by presence in the reaction mixture of either strand of a target nucleic acid that can be a long polynucleotide comprising the target sequence at its 5′-end. The target sequence must be single-stranded to trigger the amplification process. If target sequence is a fragment of long double-stranded polymer, it needs to be rendered single-stranded (e.g. denatured) prior the amplification, and results of FIG. 8 illustrate that. The amplified target material was detected by monitoring EvaGreen™ fluorescence every 10 seconds in ‘Green’ channel of the instrument set up at ‘Gain 6’. Reactions contained forward and reverse primers (SEQ ID NO:1, 2), both at 2 micromolar concentration, 0.15 Units per microliter of Vent(exo-) DNA polymerase and 10 thousand molecules of M13mp18 cloning vector prepared as described in Example 2. This long nucleic acid incorporated the target sequence of oligonucleotide SEQ ID NO:4, but it cannot trigger the amplification process until it is melted at least ones as shown in FIG. 8. The double-melt approach (time/temperature profile C, FIG. 8) insures highest concentration of the target sequence in single-stranded form resulting, as anticipated, in earlier detection of the isothermally-amplified target duplex (FIG. 8).

Example 3.5. The isothermal amplification method of FIG. 2 can be incorporated into and accelerate many target amplification technologies presently known in the art. PCR is one of the examples, and the results shown in FIGS. 9A and 9B illustrate that. Reaction composition in this set of experiments was the same as described in FIG. 7 and working Example 3.3 with one difference. Circular double-stranded cloning vector M13mp18, linearized as described in Example 2, was used as a target sequence in the target-titration experiments of FIG. 9A. PCR was performed by using a conventional PCR time/temperature profile and the same profile, but incorporating incubation of the reaction mixture at primer-cycling temperature of 73° C. for 20 seconds during each PCR cycle. Calculation based on linear slope coefficients of FIG. 9B indicates that the addition of this isothermal amplification step increases the PCR amplification power from 2, i.e. theoretical maximum of conventional PCR, to 2.5. If desired, the PCR amplification power value in this reaction can be increased by using a longer time for the reaction exposure at the primer-cycling temperature than that shown in FIG. 9.

Example 3.6. FIGS. 11A and 11B illustrate PCR application of methods shown in FIGS. 1 and 3 wherein only one primer of the primer pair is involved in an isothermal cycling stage of the reaction. Reaction mixtures comprised forward primer SEQ ID NO:14 (2 μM), reverse primer SEQ ID NO:16 (200 nM) and 5′-cleavable FRET-probe SEQ ID NO:15 (600 nM concentration in the reaction mixture). Sequences of these oligonucleotide components are shown in FIG. 10. A combination of two enzymes, Vent(exo-)™ and Taq (GenScript™, Cat. NO: E00007) DNA polymerases were used in this set of experiments at concentrations of 0.1 and 0.125 Units per microliter, respectively. M13mp18 double-stranded DNA (prepared as described in Example 2) was used as a target sequence in the target-titration experiments of FIG. 11A. Similar to the experiment of FIG. 9A, PCR was performed by using a conventional PCR time/temperature profile and the same profile but incorporating incubation of the reaction mixture at primer-cycling temperature of 85° C. for 40 seconds during each PCR cycle. Although only one primer SEQ ID NO:14 is isothermally cycling in this PCR method, the calculation based on linear slope coefficients of FIG. 11B indicates that the addition of this isothermal amplification step increases the amplification power from 2 (commonly observed in conventional PCR) to 3.3. The increase in amplification power, due to the cycling approach of the invention (FIGS. 1-3), can cause noticeable (see FIG. 9A) or even significant reduction (see FIG. 11A) in the PCR cycle number that is necessary to amplify and detect the same target load compared to conventional PCR. Unlike the method scheme of FIG. 2, the methods of FIGS. 1 and 3 may be less constrained regarding the length and nucleotide composition of the target amplicon between the primer sequences and their respective binding sites. For example, the primer design shown in FIG. 10 accommodates a 14-mer target sequence portion between the primers' binding sites for the FRET-probe binding, while maintaining good isothermal cycling efficiency driven by primer SEQ ID NO:14, and thus substantially accelerating the PCR (FIG. 11A).

### REFERENCES CITED, AND INCORPORATED HEREIN BY REFERENCE THERETO FOR THEIR RESPECTIVE RELEVANT TEACHINGS

Ausubel F. M et al, eds. (1993) Current Protocols in Molecular Biology, Vol. 1, Chapter 2, Section I, John Wiley & Sons, New York.
Beaucage S. L., Caruthers M. H. (1981) Tetrahedron Lett. V. 22, 1859-1862.
Boom W. R., Henriette M. A., Kievits T., Lens P. F. (1993) U.S. Pat. No. 5,234,809.
Breslauer K. J. et al (1986) Proc. Natl. Acad. Sci. USA, V. 83, 3746-3750.
Caruthers M. H., Matteucci M. D. (1984) U.S. Pat. No. 4,458,066.
Cleuziat P. and Mandrand B. (1998) U.S. Pat. No. 5,824,517.
Davey C. and Malek L. T. (2000) U.S. Pat. No. 6,063,603.
Didenko V. V. (2001) BioTechniques, V. 31, 1106-1121.
Duck P., Bender R., Crosby W., Robertson J. G. (1989) U.S. Pat. No. 4,876,187.
Eckstein F., ed., (1991) Oligonucleotides and Analogs: A Practical Approach. Oxford University Press, New York.
Eftink M. R. (1991) Fluorescence quenching: theory and applications. In Lakowicz J. R. (ed.), Topics in Fluorescence Spectroscopy. Plenum Press, New York, V. 2: 53-126.
Forster T. (1965) Delocalized excitation and excitation transfer. In Sinanoglu, O. (ed.), Modern Quantum Chemistry, Istanbul Lectures, part III. Academic Press, New York, 93-137.
Fraiser M. S. et al (1997) U.S. Pat. No. 5,648,211.
Gait M. J., ed., (1984) Oligonucleotide Synthesis: A Practical Approach, IRL Practical Approach Series, IRL Press, Oxford.
Ge W. et al (2012) US Patent Application Publication 2012/0058481.
Hedgpeth J., Afonina I. A., Kutyavin I. V., Lukhtanov E. A., Belousov E. S., Meyer, Jr. R. B. (2010) U.S. Pat. No. 7,794,945.
Kawase Y. et al (1986) Nucleic Acids Res., V. 14, 7727-7736.
Kornberg A., and Baker T. (1992) DNA Replication, Second Edition, W. H. Freeman and Company, New York.
Kurn N. (2001) U.S. Pat. No. 6,251,639.
Kutyavin I. V., Milesi D., Hoekstra M. F. (2007) U.S. Pat. No. 7,252,940.
Kutyavin I. V. (2007a) PCT patent application, WO/2007/127999.
Kutyavin I. V. (2007b) PCT patent application, WO/2007/127992.
Kutyavin I. V. (2012) U.S. Pat. No. 8,143,006.
Kutyavin I. V. (2015) U.S. Pat. No. 9,121,056.
Kutyavin I. V. (2018) U.S. Pat. No. 9,914,963.
Lebedev Y. et al (1996) Genet. Anal., V. 13, 15-21.
Lehninger A. L. (1975) Biochemistry, 2nd edition. New York, Worth Publishers, Inc.
Lizardi P. (1998) U.S. Pat. No. 5,854,033.
Lizardi P. (2001) U.S. Pat. No. 6,210,884.
Lokhov, S. G. et al. (1992) Bioconjugate Chem., V. 3, No. 5, 414-419.
Mackay I. M. et al (2002) Nucleic Acids Res., V. 30, 1292-1305.
Mackay J., Landt 0. (2007) Methods Mol. Biol., V. 353, 237-262.
Martin F. H. et al (1985) Nucleic Acids Res., V. 13, 8927-8938.
McPherson M. J. et al, eds (1991) PCR: A Practical Approach. IRL Press,
Oxford.
McPherson M. J. et al, eds (1995) PCR2: A Practical Approach. IRL Press, Oxford.
Millar D. S., Melki J. R., Grigg G. W. (2006) PCT patent application, WO 2006/125267.
Miller S. A., Dykes D. D., Polesky H. F. (1988) Nucleic Acids Res., V. 16, 1215.
Mullis K. B. (1987) U.S. Pat. No. 4,683,202.
Mullis K. B. et al (1987) U.S. Pat. No. 4,683,195.
Nazarenko I. A. et al (1999) U.S. Pat. No. 5,866,336.
Nelson J. R. et al (2008) PCT patent application, WO/2008/086381.
Notomi T., Hase T. (2002) U.S. Pat. No. 6,410,278.
Rabbani E. et al (2016) U.S. Pat. No. 9,353,405.
Robelek R., Niu L., Schmid E. L., Knoll W. (2004) Anal. Chem., V. 76, 6160-6165.
Sambrook J., Fritsch E. F. and Maniatis T. (1989) Molecular Cloning: A Laboratory Manual, 2nd Edition. Cold Spring Harbor Lab. Cold Spring Harbor, N.Y.
Seela et al., (1992) Nucleic Acids Research, vol. 20, 55-61.
Van Ness J. et al (2003) US Patent Application Publication 2003/0138800.
Vincent M., Xu Y. and Kong H. (2004) EMBO reports, V. 5, 795-800.
Walker G. T. (1998) U.S. Pat. No. 5,712,124.
Walker G. T., Little M. C., and Nadeau J. G. (1993) U.S. Pat. No. 5,270,184.
Walsh P. S., Metzger D. A., and Higuchi R. (1991) Biotechniques, V. 10, 506-513.

1. A method for isothermally-accelerated amplification of a target nucleic acid sequence, comprising:
incubating a reaction mixture at a primer-cycling temperature, the reaction mixture sufficient to support DNA synthesis and containing DNA polymerase activity, complementary first and second target sequence template strands, a first primer P1 complementary to a 3′-terminal portion of the first target sequence template strand, a second oligonucleotide primer P2 complementary to a 3′-terminal portion of the second target sequence template strand, the P1 and P2 primers each present in excess molar concentration relative to the first and second target sequence template strands, respectively;
hybridizing, during the incubating, P1 and P2 primers to the first and the second target sequence template strands, respectively;
extending, during the incubating, the hybridized P1 and P2 primers to produce second and first target sequence template strands, respectively;
denaturing, during the incubating, the first and the second target sequence template strands to provide the first and the second target sequence template strands in P1- and P2-primable form, respectively; and
cyclically repeating, during the incubating, the hybridizing, extending and denaturing steps for the P1 and P2 primers isothermally at the primer-cycling temperature to provide isothermal P1 and P2 primer-driven cycling, wherein in each consecutive P1 and P2 isothermal cycle, at least some of the respective second and first target sequence template strands produced in and accumulated over all prior isothermal cycles serve as additional second and first target sequence template strands, to provide for isothermally-accelerated amplification of the target nucleic acid sequence.

2. The method of claim 1, wherein the isothermal cycles for the P1 and P2 primers are symmetric, or substantially symmetric, such that the number of first and second target sequence template strands produced and accumulated is equal or substantially equal.

3. The method of claim 1, wherein isothermal cycles for the P1 and P2 primers are, at least to some extent asymmetric, such that the number of first and second target sequence template strands produced and accumulated at one or more incubation times during the reaction is not equal.

4. The method of claim 3, comprising increasing or decreasing the asymmetry by varying the relative concentrations of the P1 and the P2 primers.

5. A method for producing multiple copies of a target nucleic acid sequence, comprising:
incubating a reaction mixture at a P1 primer-cycling temperature (P1-PCT), the reaction mixture sufficient to support DNA synthesis and containing DNA polymerase activity, a first target sequence template strand, a first primer P1 complementary to a 3′-terminal portion of the first target sequence template strand and present in excess molar concentration relative to the first target sequence template strand;
hybridizing, during the incubating at the P1-PCT, a P1 primer to the first target sequence template strand;
extending, during the incubating at the P1-PCT, the hybridized P1 primer to produce a complementary second target sequence template strand having a P2 primer-binding site at a 3′-terminal portion thereof;
denaturing, during the incubating at the P1-PCT, the first and the second target sequence template strands to provide the first and the second target sequence template strands in P1- and P2-primable form, respectively; and
cyclically repeating, during the incubating, the hybridizing, extending and denaturing steps isothermally at the P1-PCT, to provide isothermal P1 primer-driven cycling to isothermally produce multiple copies of the second target sequence template strand in P2-primable form.

6. The method of claim 5, wherein the reaction mixture contains a second primer P2 complementary to the 3′-terminal portion of the second target sequence template strand and present in excess molar concentration relative to the second target sequence template strand, and wherein the method comprises:
incubating the reaction mixture at the P1-PCT;
hybridizing, during the incubating at the P1-PCT, P1 and P2 primers to the first and the second target sequence template strands, respectively;
extending, during the incubating at the P1-PCT, the hybridized P1 and P2 primers to produce second and first target sequence template strands, respectively;
denaturing, during the incubating at the P1-PCT, the first and the second target sequence template strands to provide the first and the second target sequence template strands in P1- and P2-primable form, respectively; and
cyclically repeating, during the incubating, the hybridizing, extending and denaturing steps isothermally at the P1-PCT to provide isothermal P1 and P2 primer-driven cycling, wherein in each consecutive P1 and P2 isothermal cycle, at least some of the respective second and first target sequence template strands produced in and accumulated over all prior isothermal cycles serve as additional second and first target sequence template strands, to provide for isothermally-accelerated amplification of the target nucleic acid sequence.

7. The method of claim 6, wherein the P1 and P2 isothermal cycles are symmetric, or substantially symmetric, such that the number of first and second target sequence template strands produced and accumulated is equal or substantially equal.

8. The method of claim 6, wherein the P1 and P2 isothermal cycles are, at least to some extent, asymmetric, such that the number of first and second target sequence template strands produced and accumulated at one or more incubation times during the reaction is not equal.

9. The method of claim 8, comprising increasing or decreasing the asymmetry by varying the relative concentrations of the P1 and the P2 primers.

10. The method of claim 5-9, wherein the reaction mixture contains a second primer P2 complementary to the 3′-terminal portion of the second target sequence template strand and present in excess molar concentration relative to the second target sequence template strand, and wherein the reaction further comprises, after the repeating to provide the isothermal P1 primer-driven cycling,
incubating the reaction mixture at a P2 primer hybridization and extension temperature (P2-PHET) lower than the P1-PCT;
hybridizing, during the incubating at the P2-PHET, P2 primers to the second target sequence template strands produced at the P1-PCT; and
extending, during the incubating at the P2-PHET, the hybridized P2 primers to produce complementary first target sequence template strands hybridized to the second target sequence template strands produced at the P1-PCT.

11. The method of claim 10, comprising, after extending at the P2-PHET, incubating the reaction mixture at the P1-PCT.

12. The method of claim 11, comprising alternating the incubation temperature between the P1-PCT and the P2-PHET to provide alternating P1-PCT and P2-PHET stages, and wherein in each consecutive stage at least some of the respective second and first target sequence template strands produced in and accumulated over all prior stages serve as additional second and first target sequence template strands, to provide for isothermally-accelerated amplification of the target nucleic acid sequence.

13. The method of claim 12, wherein after extending at the P2-PHET, incubating the reaction mixture at the P1-PCT denatures the hybridized template strands produced at the P2-PHET to provide the first and the second target sequence template strands in P1- and P2-primable form, respectively.

14. The method of claim 12 or 13, wherein alternating the reaction temperature between the P1-PCT and the P2-PHET to provide alternating P1-PCT and P2-PHET stages comprises, before or after incubating at the P1-PCT, incubating at a denaturation acceleration temperature greater than the P1-PCT to facilitate denaturation of the hybridized template strands produced at the P2-PHET.

15. A method for isothermally-accelerated amplification of a target nucleic acid sequence, comprising:
incubating a reaction mixture at a P1-primer-cycling temperature (P1-PCT), the reaction mixture sufficient to support DNA synthesis and containing DNA polymerase activity, complementary first and second target sequence template strands, a first primer P1 complementary to a 3′-terminal portion of the first target sequence template strand, a second oligonucleotide primer P2 complementary to a 3′-terminal portion of the second target sequence template strand, the P1 and P2 primers each present in excess molar concentration relative to the first and second target sequence template strands, respectively;
hybridizing, during the incubating at the P1-PCT, a P1 primer to the first target sequence template strand;
extending, during the incubating at the P1-PCT, the hybridized P1 primer to produce a complementary second target sequence template strand having a P2 primer-binding site at a 3′-terminal portion thereof;
denaturing, during the incubating at the P1-PCT, the first and the second target sequence template strands to provide the first and the second target sequence template strands in P1- and P2-primable form, respectively; and
repeating, during the incubating, the hybridizing, extending and denaturing steps isothermally at the P1-PCT, to provide isothermal P1 primer-driven cycling to isothermally produce multiple copies of the second target sequence template strand in P2-primable form;
incubating, after the P1 primer-driven cycling, the reaction mixture at a P2 primer hybridization and extension temperature (P2-PHET) lower than the P1-PCT;
hybridizing, during the incubating at the P2-PHET, P2 primers to the second target sequence template strands produced at the P1-PCT;
extending, during the incubating at the P2-PHET, the hybridized P2 primers to produce complementary first target sequence template strands hybridized to the second target sequence template strands produced at the P1-PCT;
incubating, after extending at the P2-PHET, the reaction mixture at the P1-PCT; and
alternating the reaction temperature between the P1-PCT and the P2-PHET to provide alternating P1-PCT and P2-PHET stages, and wherein in each consecutive stage at least some of the respective second and first target sequence template strands produced in and accumulated over all prior stages serve as additional second and first target sequence template strands, to provide for isothermally-accelerated amplification of the target nucleic acid sequence.

16. The method of claim 15, wherein after extending at the P2-PHET, incubating the reaction mixture at the P1-PCT denatures the hybridized template strands produced at the P2-PHET to provide the first and the second target sequence template strands in P1- and P2-primable form, respectively.

17. The method of claim 15 or 16, wherein alternating the reaction temperature between the P1-PCT and the P2-PHET to provide alternating P1-PCT and P2-PHET stages comprises, before or after incubating at the P1-PCT, incubating at a denaturation acceleration temperature greater than the P1-PCT to facilitate denaturation of the hybridized template strands produced at the P2-PHET.

18. The method of any one of claims 1-17, present as an isothermal acceleration step of a PCR reaction.

19. A PCR reaction comprising at least one cycle having an isothermal amplification step according to claims 1-17.

20. The method of any one of claims 1-19, wherein the primer(s) that provide isothermal primer-driven cycling are used at a reaction concentration greater than 200 nanomolar.

21. The method of any one of claims 1-19, wherein the P1 or the P2 primer or both primer sequences incorporate at least one DNA polymerase-compatible structural modification.

22. The method of any one of claims 5, 10-17, wherein the P1 primer incorporates at least one polymerase-compatible duplex-stabilizing structural modification.

23. The method of any one of claims 1-22, wherein the amplification products are detected.

24. The method of claim 23, wherein the amplification and detection reactions are performed simultaneously, in real time.

25. The method of claim 24, further comprising determining the amount of the target nucleic acid in or from a sample.

26. The method of claim 25, wherein the reaction mixture further comprises a detectable label.

27. The method of claim 26, wherein the detectable label comprises a fluorescent label.

28. The method of claim 27, wherein the reaction mixture comprises an oligonucleotide probe labeled with two dyes that are in FRET interaction, and wherein duplex formation of the probe with products of extension of first or second primers disrupts FRET resulting in a detectable signal.

29. The method of claim 27, wherein at least one of the P1 and P2 primers is labeled with two dyes that are in FRET interaction, and wherein hybridization and extension of the primer during the amplification disrupts FRET resulting in a detectable signal.

30. The methods of claims 1-4, 6-9 and 18-29, wherein the distance, in nucleotides, between the 5′ end of first primer binding site on the first strand and the 5′ end of the second primer binding site on the second strand within the target sequence template strands is less than 20, less than 15, less than 10, less than 5, less than 4, less than 3, less than 2, 1, or 0, or is a value in the range of 0-20, or in any subrange thereof.

31. The method of any one of claims 1-30, wherein the DNA polymerase activity is provided by one of Vent(exo-) and Deep Vent(exo-) DNA polymerases or a combination thereof.

32. An isothermally-accelerated amplification kit, comprising at least two oligonucleotide primers each complementary to a respective different primer binding site of a target sequence, wherein a first oligonucleotide primer is complementary to a first primer binding site on a first strand of the target sequence, wherein the second oligonucleotide primer is complementary to a second primer binding site on a second, complementary strand of the target sequence to define an amplicon bracketed by the first and second primers, and wherein, relative to the target sequence, the sequences and relative positions of the first and second primer binding sites on the target sequence are such that thermal stability of the primers and their extension products, when hybridized to the target sequence, provides for isothermal cycles of primer binding, primer extension, and primer extension product denaturation.

33. The kit of claim 32, wherein the distance, in nucleotides, between the 5′ end of first primer binding site on the first strand and the 5′ end of the second primer binding site on the second strand is less than 20, less than 15, less than 10, less than 5, less than 4, less than 3, less than 2, 1, or 0, or is a value in the range of 0-20, or in any subrange thereof.

34. The kit of claim 33, wherein the distance is 0 to 3 nucleotides.

35. An isothermally-accelerated amplification kit, comprising at least two oligonucleotide primers each complementary to a respective different primer binding site of a target sequence, wherein a first oligonucleotide primer is complementary to a first primer binding site on a first strand of the target sequence, wherein the second oligonucleotide primer is complementary to a second primer binding site on a second, complementary strand of the target sequence to define an amplicon bracketed by the first and second primers, and wherein, relative to the target sequence, the distance, in nucleotides, between the 5′ end of first primer binding site on the first strand and the 5′ end of the second primer binding site on the second strand is less than 20, less than 15, less than 10, less than 5, less than 4, less than 3, less than 2, 1, or 0, or is a value in the range of 0-20, or in any subrange thereof.

36. The kit of claim 35, wherein the distance is 0 to 3 nucleotides.

37. The kit of claim 35 or 36, wherein, relative to the target sequence, the sequences and relative positions of the first and second primer binding sites on the target sequence are such that thermal stability of the primers and their primer extension products, when hybridized to the target sequence, provides for isothermal cycles of primer binding, primer extension, and primer extension product denaturation.
